(19) United States
(12) Patent Application Publication
(10) Pub. No.: US 2004/0009149 A1
(43) Pub. Date: Jan. 15, 2004

(54) Multimeric binding complexes

(76) Inventors: John D. Altman, Decatur, GA (US); Eugene Ravkov, Tucker, GA (US)

Correspondence Address: FISH & RICHARDSON PC, 225 FRANKLIN ST, BOSTON, MA, 02110, US

(21) Appl. No.: 10/376,887

(22) Filed: Feb. 27, 2003

Related U.S. Application Data
(60) Provisional application No. 60/360,724, filed on Feb. 27, 2002.

(51) Int. Cl. A61K 38/21; C07K 14/52

(52) U.S. Cl. 424/85.2; 424/85.7; 424/85.6; 424/85.5; 530/351

(57) ABSTRACT

The invention provides multimeric receptor-binding complexes, including chemokine tetramers, useful for recognizing and binding receptors bound to the surface of a wide variety of cells. The binding complexes are useful for identifying and isolating cells according to their specific receptors, screening for cells having a specific receptor or constellation of receptors, and introducing exogenous molecules (e.g., nucleic acids and toxins) into cells. Methods of producing the complexes and other uses are also described.

The present application claims the benefit of U.S. S. No. 60/360,724, which was filed on Feb. 27, 2002. The contents of that provisional application are hereby incorporated by reference in their entirety.

### TECHNICAL FIELD

This document describes multimers, including tetramers, that contain signaling molecules such as growth factors, cytokines, neurotransmitters, and hormones, and methods for using those multimers to affect a biological cell.

### BACKGROUND

The cells that are referred to as “T cells” are a subset of lymphocytes, so-named because they originate in the thymus and undergo thymic processing. T cells are primarily involved in cell-mediated immune reactions, and they help control B cell development. As such, T cells constitute a major part of the body's immune defenses against pathogens, and they have been implicated in the rejection of cancerous cells. T cells can be further classified depending on, for example, the types of receptors they express. T cells that express CD4 receptors are mainly helper T cells, and T cells that express CD8 receptors are mainly cytotoxic or suppressor T cells. CD4- and CD8-positive T cells can, in addition, be either naïve T cells or memory T cells. Naïve T cells resemble one another and are capable of becoming memory T cells. Memory T cells are unique from one another and not only combat the spread of pathogens (“effector” memory T cells), but also guard against subsequent infections (“central” memory T cells).

Many types of cells associated with the immune system, including T cells, B cells, and dendritic cells, express receptors for chemokines, which are peptides that mediate chemotaxis and are believed to play a role in autoimmune and inflammatory diseases. The chemokine receptors have seven transmembrane domains and are coupled to G proteins. Chemokine expression is induced at sites of inflammation, where these proteins function to recruit inflammatory cells that mediate tissue destruction, and expression is constitutive in lymphoid tissues (e.g., lymph nodes and the spleen), where it orchestrates the response to antigens, including autoantigens, that drive autoimmune disease.

More specifically, chemokine receptors are defined by their ability to generate a biochemical signal when bound by a member of the chemokine superfamily or by a chemotactic cytokine. To date, at least 18 human proteins have been found to meet this definition. These proteins are designated CXCR1 through CXCR5; CCR1 through CCR11; XCR1; and CX3CR1, based on their preference for specific chemokines (see Murphy et al., Pharmacol. Rev. 52:145-176, 2000). In addition to being expressed in mammals, chemokine receptor-like sequences have been identified in birds (Gupta et al., Biochem. Mol. Biol. Int. 44:673-681, 1998) and fish (Daniels et al., J. Leukol. Biol. 65:684-690, 1999), but they have not been found in invertebrates. The major biological function shared by these chemokines is in leukocyte trafficking and dependent processes, such as immune surveillance, innate and adaptive immune responses, and various forms of pathological inflammation (Springer, Cell 76:301-314, 1994; Foxman et al., J. Cell Biol. 139:349-360, 1997).

### SUMMARY

The invention described herein encompasses multimeric complexes that can bind receptors, such as cell surface receptors (e.g., receptors for growth factors or cytokines, including chemokines and interleukins, neurotransmitters, and hormones) or analogs thereof. These receptor-binding complexes may be referred to herein simply as “complexes,” and they can be used in a variety of ways. The complexes can, for example, be used to detect, quantitate, characterize, or separate cells or membrane fractions bearing any given receptor, receptor complex, or analog thereof, whether naturally expressed, genetically engineered, or synthesized. Given the instructions that follow and the techniques currently available in the art, one of ordinary skill in the art can readily make a variety of complexes that bind a variety of receptors (e.g., growth factor, cytokine, chemokine, or interleukin receptors or receptor complexes (or analogs thereof)).

Methods of making the complexes and other compositions of the invention are described further below, as are methods of using them to deliver moieties (e.g., detectable labels, nucleic acids, other therapeutic agents, or toxins) to, for example, a cell that expresses the cognate receptor. For example, a complex including nerve growth factor (NGF) as the receptor-binding molecule can be used to detect an NGF receptor or deliver a moiety to an NGF receptor-bearing cell; a complex including interleukin 2 (IL-2) as the receptor-binding molecule, can be used to detect an IL-2 receptor or deliver a moiety to an IL-2 receptor-bearing cell; a complex including CCL19 as the receptor-binding molecule can be used to detect a CCL19 receptor (CCR7) or deliver a moiety to a CCR7-bearing cell; etc. In any case, the detection or delivery can be carried out for diagnostic or therapeutic reasons (particular assays and treatments are described further below). The complexes can also be used to identify therapeutic agents. For example, a potential therapeutic agent can be incorporated into the complex (in addition to, or in place of, one or more of the “receptor-binding molecules” described below), and brought into contact with a biological cell (in culture or in vivo). One can then assess the cellular response (or, if the assay is carried out in vivo, one can assess signs or symptoms of disease in the animal) to determine whether the agent exerts a desired effect. The precise parameter assessed can vary, depending on the agent tested and its expected application.

With respect to treatment, the complexes of the invention can be used in many circumstances. Wherever an affected cell type has a known, and preferably unique, cell-surface receptor, the complexes of the invention can be used to preferentially bind that receptor and thereby target a therapeutic agent to the affected cell. For example, where a cancerous cell expresses a particular growth factor receptor (e.g., an EGF receptor or a steroid receptor, such as an estrogen receptor), a complex of the invention that includes the cognate ligand (e.g., EGF, or a steroid, respectively) and a chemotherapeutic agent can be used to target that agent to the cancerous cell. Similarly, the complexes of the invention can be used to target therapeutic agents to neurons that express receptors for a given neurotransmitter. For example, a complex containing dopamine can be used to deliver an agent to dopamine target cell; a complex containing acetylcholine can be used to deliver an agent to a cholinoceptive neuron; a complex containing serotonin can be used to deliver an agent to a serotonergic neuron; etc.

While immunocytochemistry can presently be used to identify cells bearing many types of receptors, antibodies that specifically bind all of the known receptors or receptor-binding ligands are not available. The complexes described herein allow one to detect receptors in such cases (i.e., where antibodies are not available or are less than optimal). Where the specificity of the interaction between the complexes of the invention and their cognate receptor is high, the complexes can be used to target cells that express a particular cytokine receptor. This is advantageous because it may increase the specificity with which nucleic acid molecules, other therapeutic agents, or other types of molecules (such as toxins) can be delivered to a patient. In addition, when compared to the binding of monomeric ligands, the complexes of the invention may be more stable; they can, for example, have an increase in t½ that is 5-, 10-, 15-, 20-, or even 50-fold higher than for uncomplexed signaling molecules.

Although methods and materials similar or equivalent to those described herein can be used in the practice or testing of the present invention, useful methods and materials are described below. The materials, methods, and examples are illustrative only and not intended to be limiting. Other features and advantages of the invention will be apparent from the drawings, the following detailed description, and from the claims.

### BRIEF DESCRIPTION OF THE DRAWINGS

FIG. 1 represents flow cytometry data collected following CCL19-chemokine staining of CD20⁺ cells (representative of B cells) and CD16⁺CD56⁺ lymphocytes (representing NK cells). “CCL19-tet PE” is an abbreviation denoting CCL19-tetramers tagged with phycoerythrin.

FIG. 2 represents flow cytometry data collected following CCL19-chemokine staining on CD3⁺CD4⁺ cells and CD3⁺CD8⁺ cells.

FIG. 3 represents flow cytometry data collected from CD3⁺CD8^{+ and CD}3⁺CD8⁻ cells; 1 μg of anti-CCL19 mAb blocked CCL19-chemokine staining.

FIG. 4 represents flow cytometry data from CD3⁺CD8⁺ and CD3⁺CD8⁻ (CD3⁺CD4⁺) cells; heparin blocked CCL19-chemokine staining. “CCL19-tet APC” is an abbreviation denoting CCL19-tetramers tagged with allophycocyanin.

FIG. 5 represents flow cytometry data of CD3⁺CD8⁺ and CD3⁺CD4⁺ cells that were preincubated with increasing concentrations of CCR7 mAb. Preincubation abrogated CCL19-chemotetramer binding.

FIG. 6 represents flow cytometry data from CD3⁺CD8⁺ cells that were stained simultaneously with a CCL19 chemotetramer and CCR7 mAb. When both reagents are added to the cells at the same time, they bind to the same subset of cells.

FIG. 7 represents flow cytometry data from CD3⁺CD8⁻ cells that were stained simultaneously with a CCL19 chemotetramer and CCR7 mAb. When both reagents are added to the cells at the same time, they bind to the same subset of cells.

FIG. 8 represents flow cytometry data from CD3⁺CD8⁺ cells costained with a CCL19-chemotetramer and a panel of markers of naïve and antigen-experienced CD8⁺ T cells. The markers include CD11a, which is a marker for antigen-experienced cells, and CD45RA and CD62L, which are markers for naïve T cells.

FIG. 9 represents flow cytometry data from a phenotypic analysis performed on A2/CMV⁻, B8/EBV⁻ and A2/flu-specific CD8⁺ T cells from different donors. Cells were stained with CCL19-tetramers labeled with phycoerythrin (CCL19-tet PE) and a panel of cell surface markers.

FIG. 10 represents flow cytometry data from a phenotypic analysis performed on A2/CMV-specific CD8⁺ T cells from donor #20. Cells were co-stained with CCL19-tetramers labeled with phycoerythrin (CCL19-tet PE) and a panel of cell surface markers labeled with FITC.

FIG. 11 represents flow cytometry data from a phenotypic analysis performed on B8/EBV.RAK-specific CD8⁺ T cells from donor #22. Cells were co-stained with CCL19-tetramers labeled with phycoerythrin (CCL19-tet PE) and a panel of cell surface markers labeled with FITC.

FIG. 12 represents flow cytometry data from a phenotypic analysis performed on A2/flu-specific CD8⁺ T cells from donor #38. Cells were co-stained with CCL19-tetramers labeled with phycoerythrin (CCL19-tet PE) and a panel of cell surface markers labeled with FITC.

FIG. 13 represents flow cytometry data from a phenotypic analysis performed on B8/EBV.RAK-specific CD8⁺ T cells from donor #42. Following stimulation with a cognate peptide (see FIG. 24), intracellular cytokine staining for IFNγ was performed. The cells were also stained with CCL19-tetramers labeled with phycoerythrin (CCL19-tet PE).

FIG. 14 represents flow cytometry data from a phenotypic analysis performed on B8/EBV.FLR-specific CD8⁺ T cells from donor #42. Following stimulation with a cognate peptide (see FIG. 24), intracellular cytokine staining for IFNγ was performed. The cells were also stained with CCL19-tetramers labeled with phycoerythrin (CCL19-tet PE).

FIG. 15 represents flow cytometry data from a phenotypic analysis performed on A2/flu-specific CD8⁺ T cells from donor #42. Following stimulation with a cognate peptide (see FIG. 24), intracellular cytokine staining for IFNγ was performed. The cells were also stained with CCL19-tetramers labeled with phycoerythrin (CCL19-tet PE).

FIG. 16 represents flow cytometry data from a phenotypic analysis performed on B8/EBV.FLR-specific CD8⁺ T cells from donor #22. Following stimulation with a cognate peptide (see FIG. 24), intracellular cytokine staining for IFNγ was performed. The cells were also stained with CCL19-tetramers labeled with phycoerythrin (CCL19-tet PE).

FIG. 17 represents flow cytometry data from a phenotypic analysis performed on B8/EBV.RAK-specific CD8⁺ T cells from donor #22. Following stimulation with a cognate peptide (see FIG. 24) for six hours, intracellular cytokine staining for IFNγ was performed. The cells were also stained with CCL19-tetramers labeled with phycoerythrin (CCL19-tet PE).

FIG. 18 represents data from a FACS sorting experiment. Freshly prepared PBMCs from donor #22 were stained with the B8/EBV.RAK tetramer and the CCL19-chemotetramer and then FACS sorted into B8/EBV.RAK⁺CCR7⁺ and B8/EBV.RAK⁺CCR7⁻ subsets. The figure illustrates the quality of the sorting procedure and the gating strategy used.

FIG. 19 represents data from a FACS sorting experiment. The CCR7^{+ and CCR}7⁻ sorted cells described in FIG. 18 were stimulated for six hours with peptide-pulsed autologous B-LCL, and then examined for their ability to produce IFNγ.

FIG. 20 represents flow cytometry data from human PBMCs stained with APC-labeled huMIP-3β tetramer and a cocktail of labeled monoclonal antibodies specific for CD45RA, CD62L, and CD4. Contour plots display cells gated on CD4⁺ lymphocytes. Histograms of CD62L staining of cells falling within each of the labeled quadrants is also displayed.

FIG. 21 represents flow cytometry data of human PBMC stained with APC-labeled huMIP-3β tetramer and a cocktail of labeled monoclonal antibodies specific for CD45RA, CD62L, and CD8. Contour plots display cells gated on CD4⁺ lymphocytes. Histograms of CD62L staining of cells falling within each of the labeled quadrants is also displayed.

FIG. 22 represents flow cytometry data illustrating staining patterns for CCR7, CD45RA, and CD62L on CD4⁺ lymphocytes (top row) and on CD8⁺ lymphocytes (bottom row) (Sallusto et al., Nature 401:708-712, 1999).

FIG. 23 represents flow cytometry data presented in contour plots. Human PBMCs were stained with the MIP-3β tetramer and antibodies against CD4, CD8 and CCR7.

FIG. 24 is a Table listing representative chemokines and their receptors.

FIG. 25 is a Table (in two parts) showing phenotypic analyses of antigen-specific CD8⁺ T cells.

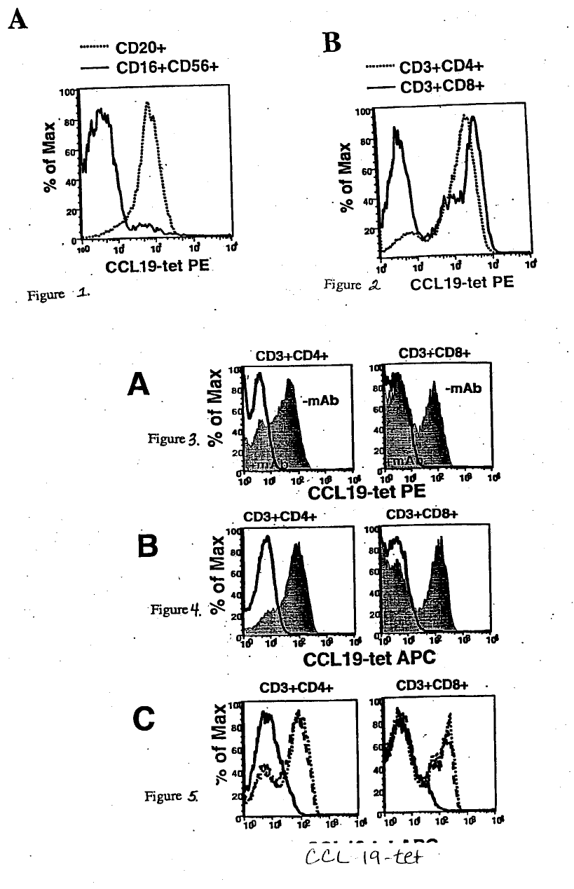

### DETAILED DESCRIPTION

Some of the compositions described herein are, or include, complexes that contain at least one (e.g., one, two or more) receptor-binding molecules, at least one multivalent binding partner, and a linker that links the receptor-binding molecules to the multivalent binding partner. The bonds formed between the members of the complex can be covalent or non-covalent. For example, the receptor-binding molecules can be proteinaceous and can be bound by way of a peptide bond to the linker, which may then be modified to facilitate interaction with the multivalent binding partner. Two or more linkers, either as initially made or as subsequently modified (modification is discussed further below), associate (again, by forming covalent or non-covalent attachments) with a multivalent binding partner, which can, but does not necessarily, carry a detectable marker. More generally, the complex can be formed by way of a chemical reaction between one or more of the components; by way of peptide bond formation; by way of epitope receptor binding; or by way of hapten receptor binding, where a hapten is linked to a monomeric component of the complex.

In addition to the complex, compositions containing just a single receptor-binding molecule and a linker are within the scope of the present invention. For example, the invention features a receptor-binding molecule that is, or that includes, an amino acid sequence that is joined to (e.g., fused to) a proteinaceous linker (e.g., a linker containing an amino acid sequence that can be biotinylated, glycosylated, farnesylated, or phosphorylated). More specifically, the receptor-binding molecule can be any of the molecules described below. However, and as noted below, regardless of the configuration (whether in a complex or simply bound to a linker), the receptor-binding molecule cannot contain major histocompatibility complex (MHC) protein subunits that have homogeneous populations of peptides bound in the antigen presentation site. Suitable linkers are also described further below.

Receptor-binding molecules: As implied above, the number of receptor-binding molecules in a given complex can vary; each complex can have 1, 2, 3, 4, or more receptor-binding molecules. While complexes may contain more than ten receptor-binding molecules, it is expected that ten or fewer (e.g., 1, 2, 4, 6, or 8) will be more commonly used. These molecules may be identical or non-identical and they may be from the same or different species. For example, one or more (or all) of the receptor-binding molecules can be from a mammal, a bird, a fish, a reptile, an amphibian, or an insect. Mammalian receptor-binding molecules can be human or from a non-human primate (such as a monkey, chimpanzee or gorilla); from a domesticated animal (such as a dog, cat, horse, cow, pig, sheep, or goat); or from an animal commonly used in laboratory or pre-clinical studies (such as a mouse, a rat, a hamster, a guinea pig, or a rabbit). Non-mammalian animals are also often used in laboratory and pre-clinical studies, and the complexes described here can be used to develop therapeutic agents in those animals (complexes containing these types of receptor-binding molecules may be used for other purposes (e.g., they may be useful in screening assays or in therapeutic regimes)). Accordingly, the complexes of the invention can contain receptor-binding molecules from birds (e.g., a chicken), frogs (e.g., Xenopus), zebrafish, nematodes (e.g., C. elegans), or flies (e.g., Drosophila).

The receptor-binding molecules can have sequences that are naturally occurring or mutant. For example, a complex can contain a biologically active fragment or other mutant (e.g., a mutant containing a number of substituted residues) of a receptor-binding molecule. Biologically active fragments or other mutants will retain a detectable amount of one or more of the biological activities of the corresponding wild type molecule; preferably, the fragments or other mutants will retain the ability to bind the cognate receptor. Moreover, either naturally occurring or mutant sequences can contain additional sequences at one termini or the other (e.g., sequences that improve the ease of making or using the complexes, sequences that facilitate their detection, or sequences that alter (e.g., improve) the activity of the receptor-binding molecule (e.g., sequences that improve its binding affinity or specificity).

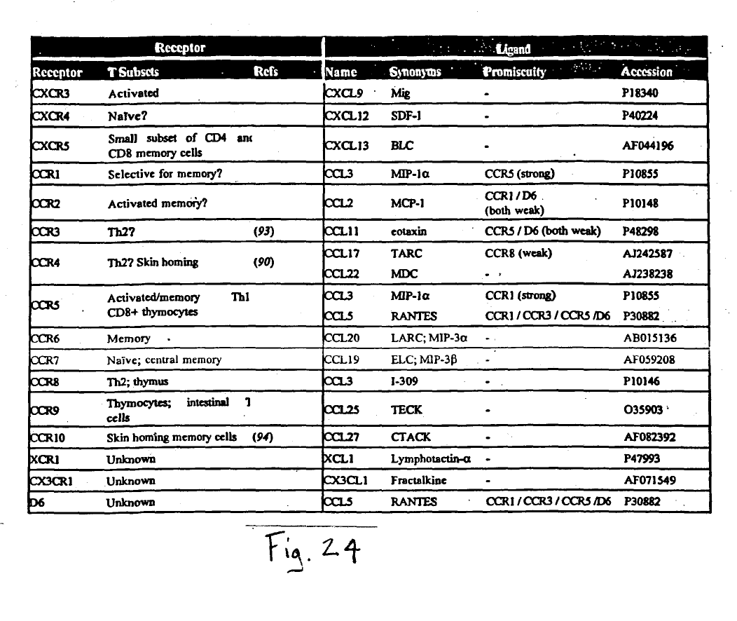

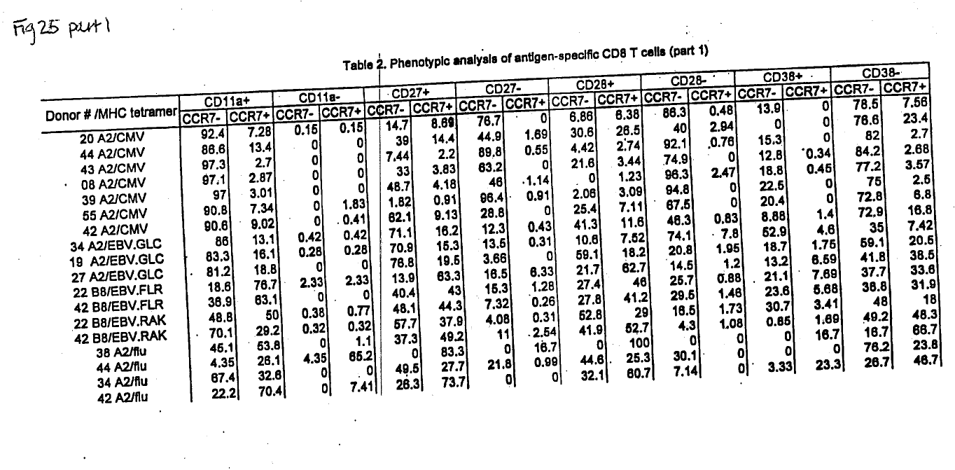

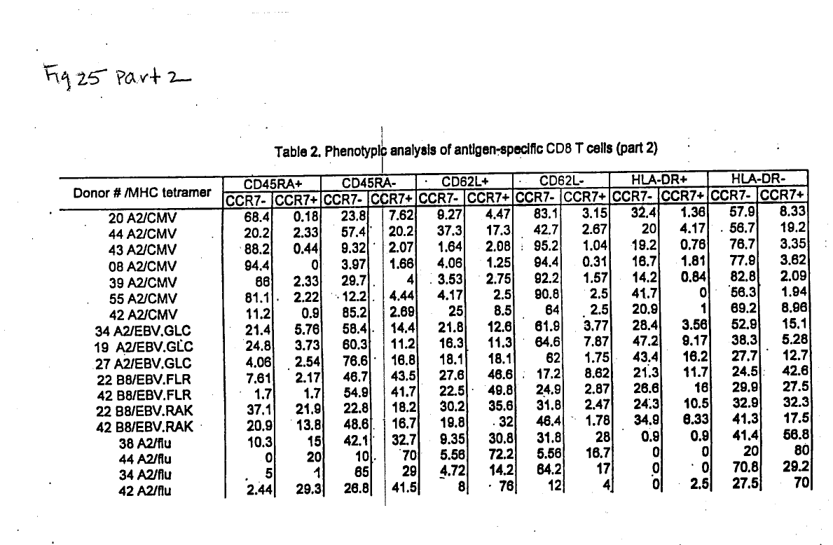

The receptor-binding molecule can be, as its name implies, any molecule that is capable of binding to a cellular receptor (with the MHC proteins above as a noted exception). For example, the receptor-binding molecule can be a signaling molecule such as a growth factor, or cytokine (e.g., a chemokine or interleukin), a neurotransmitter or hormone. While these molecules bind cell surface receptors, the receptor-binding molecule can also bind an intracellular receptor, such as a nuclear receptor. Due to their profound effect on development and adult physiology, signaling molecules have been extensively studied. Many are known and well characterized; characteristics such as their size, sequence, and receptor-binding properties are known in many cases. For example, chemokines, generally considered a subset of cytokines, constitute a large family of secreted molecules that, inter alia, stimulate chemotaxis of leukocytes (including basophils, eosinophils, lymphocytes, monocytes, and neutrophils). Chemokines include CCL19 (also known as MIP-3β or ELC), CCL20 and CXCL12, which are recognized by the chemokine receptors CCR7, CCR6 and CXCR4, respectively (see also FIG. 24). When the complex contains four receptor-binding partners, and those partners are chemokines, the complex may be referred to as a chemotetramer or chemokine tetramer.

More specifically, the invention encompasses multimeric complexes that include at least one and, more typically, at least two receptor-binding molecules, a multivalent binding partner, and a linker that joins the receptor-binding molecules to the multivalent binding partner. The receptor-binding molecules, which may be identical to one another or may vary, can be signaling molecules, such as growth factors or biologically active fragments or mutants thereof. For example, the growth factor can be a cytokine (e.g., an interleukin or chemokine). While one of ordinary skill in the art can readily determine whether a molecule is a signaling molecule (i.e., whether it is produced and secreted by a first cell or cell type and exerts an effect on a second cell or cell type, usually by specifically binding a receptor on the second cell), various particular signaling molecules may be properly placed in two or more categories. For example, CCL7 may be properly referred to as a cytokine or chemokine; IL-1 may be properly referred to as a cytokine or interleukin; erythropoietin may be properly referred to as a growth factor or a hormone; etc.

The interleukin can be IL-1, IL-2, IL-3, IL-4, IL-5, IL-6, IL-7, IL-8, IL-9, IL-10, IL-11, IL-12, IL-13, IL-15, IL-21, interferon-alpha (IFNα), interferon-beta (IFNβ), or interferon-gamma (IFNγ). The chemokine can be a member of the α subfamily and/or can bind a CXCR1, CXCR2, CXCR3, CXCR4, or CXCR5 receptor; it can be a member of the β subfamily and/or can bind a CCR1, CCR2, CCR3, CCR4, CCR5, CCR6, CCR7, CCR8, CCR9, CCR10, or CCR11 molecule. The chemokine can also be lymphotactin or another chemokine that binds a XCR1 receptor; the chemokine can also be fractalkine or can bind a CX₃CR1 receptor. For example, the chemokine can be CCL7, CCL23, CCL27, CCL28, CXCL12, CXCL14, or CXCL15.

Suitable growth factors are members of the tumor necrosis factor (TNF) family, members of the nerve growth factor (NGF) family, members of the transforming growth factor (TGF) family, members of the fibroblast growth factor (FGF) family, members of the insulin-like growth factor (IGF) family, members of the epidermal growth factor (EGF) family, or members of the platelet-derived growth factor (PDGF) family. For example, the growth factor can be TNF, EGF, TGFα, TGFβ, FGF, NGF, erythropoietin, IGF-1, or IGF-2.

The receptor-binding molecule can also be a hormone, neurotransmitter, or co-stimulatory molecule and, as with the other types of receptor-binding molecules described above, can be a biologically active fragment or other mutant (e.g., substitution mutant) of such molecules. The hormone can be a hormone produced by the adrenal gland, parathyroid gland, pituitary gland, or thyroid gland; it can also be produced by the hypothalamus, the ovary, the testicle, the pancreas, the pineal body, or the thymus. For example, the hormone can be a thyroid-stimulating hormone, a follicle-stimulating hormone, a leuteinizing hormone, prolactin, growth hormone, adrenocorticotropic hormone, antidiuretic hormone, oxytocin, thyrotropin-releasing hormone, gonadotropin-releasing hormone, growth hormone-releasing hormone, corticotropin-releasing hormone, somatostatin, dopamine, melatonin, thyroxine, calcitonin, parathyroid hormone, a glucocorticoid, a mineralocorticoid, an androgen, adrenaline, an estrogen, progesterone, human chorionic gonadotropin, insulin, glucagons, somatostatin, erythropoietin, calcitriol, atrial-natriuretic peptide, gastrin, secretin, cholecystokinin, somatostatin, neuropeptide Y, ghrelin, PYY₃₋₃₆, insulin-like growth factor-1, angiotensinogen, thrombopoietin, or leptin.

Suitable neurotransmitters include acetylcholine, dopamine, norepinephrine, serotonin, histamine, or epinephrine. The neurotransmitter can also be a neuroactive peptide (e.g., bradykinin, cholecystokinin, gastrin, secretin, oxytocin, a sleep peptide, gonadotropin-releasing hormone, beta-endorphin, enkephalin, substance P, somatostatin, prolactin, galanin, growth hormone-releasing hormone, bombesin, dynorphin, neurotensin, motilin, thyrotrop in, neuropeptide Y, leuteinizing hormone, calcitonin, or vasoactive intestinal peptide).

Suitable co-stimulatory molecules include B7-1 and B7-2.

Linkers: The linkers contained in the compositions described herein (whether part of the complex or a portion thereof (i.e., whether simply linked to a single receptor-binding molecule)) can vary greatly so long as they provide a means for attaching a receptor-binding molecule to a multivalent binding partner. The linkers may be, but are not necessarily, proteins. Moreover, they may be, but are not necessarily, proteins that can be modified by enzymes. For example, the linkers may be, or may contain, a peptide that can be modified by a biotin protein ligase (e.g., BirA), a glycosylase, a famesyl protein transferase, or a protein kinase.

The linker can be, or can contain, a BirA substrate peptide (BSP). While the compositions of the invention are not limited to those formed by any particular biochemical reaction, we explain here how BSP-containing linkers may participate in the complex. Linkers comprising BSP can be exposed to BirA, which will activate biotin to form biotin 5′ adenylate and will transfer the activated biotin to a biotin accepting protein (e.g., BSP). Thus, when a protein containing BSP is incubated with a BirA enzyme and biotin, the protein becomes biotinylated on the substrate peptide. The BSP can vary in length. For example, it can be 13, 14, 15, 16, 17, 18 or more amino acids long (Shatz, Biotechnology 11:1138-1143, 1993). Typically, however, the BSP is not expected to be more than 30 amino acids long.

The linker can also be, or include, a peptide tag, such as a Strep-tag® or Strep-tag® II, which binds directly to streptavidin or Strep-Tactin® (Schmidt et al., J. Mol Biol. 255:753-66, 1996; IBA, St. Louis, Mo.). Strep-tag® is a short peptide (8 amino acid residues) that selectively binds an engineered streptavidin called Strep-Tactin®. Strep-tag® can be genetically fused to any protein's N- or C-terminal end.

The linker can also be a protein or peptide (e.g., a peptide tag) that interacts with (e.g., binds to) a phosphate group on the multivalent binding protein. A variety of protein-protein interactions known in the art depend on the presence of a phosphorylated amino acid residue (for a review, see McMurray et al., Biopolymers, 60:3-31, 2001), and any of these protein pairs can be used to assemble the complex of the present invention. For example, an SH2 domain, such as that within the Src kinase protein, can bind a peptide tag having the amino acid sequence pTyr-hydrophilic residue-hydrophilic residue-Ile-Pro, where pTyr represents a phosphorylated tyrosine residue. More specifically, the Src kinase can bind the sequence pTyr-Glu-Glu-Ile (Songyang et al., Cell 72:767-778, 1993). Accordingly, phosphopeptide tags having this sequence can be used to link receptor-binding molecules to multivalent binding partners.

The linker can also be an antibody, such as an antibody that recognizes a sugar on a multivalent binding protein. Thus, the multivalent binding partners useful in the complexes of the invention can be glycosylated. An antibody linker can alternatively recognize an isoprenyl group, such as a famesyl or geranyl moiety on the multivalent binding protein. An antibody linker can also recognize a phosphate group or biotin on the multivalent protein. When the linker is an antibody, a receptor-binding molecule can be attached, covalently or noncovalently, to the antibody, for targeting of the complex to a cell.

The linker can also be, or include, a moiety attached directly to the receptor-binding molecule. For example, a biotin can be conjugated to the receptor-binding molecule, such as on a cysteine residue of the receptor-binding molecule. Biotin can be conjugated by exposing the receptor-binding molecule to a reagent such as 3-(N-maleimidopropionyl)biocytin (Sigma-Aldrich Corp., St. Louis, Mo.).

Biotinylated linkers will interact with multivalent binding partners that are, or that contain, avidin or streptavidin. Where the multivalent binding partner is labeled, this interaction facilitates detection, quantitation, or separation of the intact complex and any entity to which it binds (e.g., a receptor or receptor-bearing cell).

As noted above, the linkers and multivalent binding partners can be varied, and their selection can depend on how strongly bound or stable one wishes the complex to be. For example, Strep-Tag® II binds Strep-Tactin® with an affinity that is about 100 times greater than its affinity for streptavidin (IBA, St. Louis, Mo.). Thus, if one desired a weaker interaction between the receptor-binding molecule and the multivalent binding partner, the receptor molecule could be fused to Strep-Tag® II and then complexed with streptavidin. Weaker interactions may be preferable when one wishes the complex to dissociate; dissociation can facilitate the activity of a cargo molecule, such as a toxin or nucleic acid, that is delivered to a cell by way of attachment to the multivalent protein, receptor-binding molecule, or other part of the multimeric complex. To the contrary, tighter associations may be preferable when the stability of the complex is more important. For example, tighter association may be preferable when a multivalent binding partner (or other member of the complex) is fused to a detectable label, such as a fluorophore, for the purpose of labeling a cell.

In other configurations, the linker may be omitted from the complex altogether. For example, the receptor-binding molecule can be fused directly to the multivalent binding partner (e.g., a biotinylated or Strep-tag-bearing receptor-binding molecule can be fused directly to avidin, streptavidin, or Strep-Tactin®). The type of multivalent binding partner can help determine, and in some instances may dictate, whether a linker is necessary. For example, the multivalent binding partner can be an antibody (see below). In that case, the linker molecule can be an epitope that is fused to a receptor-binding molecule (in which case, the epitope will serve as the linker). Alternatively, the epitope can be part of the receptor-binding molecule itself (in which case, no linker is required). The overall function of the complex is a consideration as well. Even though a multivalent binding partner may be able to directly bind a receptor-binding molecule, a linker should be included if the direct binding interferes with the ability of the receptor-binding molecule to bind its cognate cellular receptor. For example, a linker should be used if an antibody, by binding an epitope within (rather than fused to) a receptor-binding molecule, prevents the receptor-binding molecule from binding its cognate cellular receptor.

Multivalent binding partner: The multivalent binding partner can be any moiety having binding sites for one, two, three, four or more linkers or one, two, three, four or more receptor-binding molecules. Accordingly, one will choose, for inclusion in the complex, a multivalent binding partner and a linker (or receptor-binding molecule) that associate with (e.g., bind to, reversibly or irreversibly) one another. For example, where the linker is a biotinylated substance (e.g., a biotinylated peptide), the multivalent binding partner will include at least one biotin-binding site. More specifically, biotinylated substances can be detected with avidin, streptavidin, or commercially available variants thereof (for example, Molecular Probes (Eugene, Oreg.) sells a variety of avidin and streptavidin conjugates (conjugated to, for example, fluorophores)).

The multivalent binding partner can also be an antibody, including an intact antibody, a fragment thereof, or a single chain antibody. The antibodies can be optimized (e.g., the CDRs can be optimized) or humanized by methods known in the art. As noted above, when an antibody serves as the multivalent binding partner, it can be used in complex that both contain and fail to contain a linker. When a linker is present, it can be a protein or peptide (or can include a protein or peptide) to which the antibody specifically binds. When the linker is absent, the antibody can specifically bind the receptor-binding molecule. Moreover, an antibody that is a multivalent binding partner can be an IgG, IgM, IgD, IgA, or IgE molecule. These antibody isotypes vary in their valencies; the valencies for IgG, IgM, IgD, IgA, and IgE are 2, 10, 6, 4, and 12, respectively.

The complex can be labeled so it can be more readily detected; it can be labeled directly (e.g., the multivalent binding partner can be tagged with a detectable marker, such as a fluorophor) or secondarily with immunoreagents that will specifically bind one or more of the components of the complex. In another configuration, a member of the complex (e.g., the multivalent binding partner) can be joined to a substance (e.g., an enzyme or enzyme substrate) that, upon exposure to a cognate reagent (e.g., upon exposure of an enzyme to a substrate or vice-versa) gives rise to a detectable agent. Thus, the label can vary significantly, so long as it is detectable. For example, the label can be detectable by virtue of its ability to emit energy (e.g., visible or fluorescent light or nuclear energy). For example, the label can be a fluorophor, such as fluorescein isothiocyanate (FITC), rhodamine, Texas Red, a green fluorescent protein, phycoerythrin (PE), or allophycocyanin (APC). These and other suitable labels are commercially available (from, for example, Molecular Probes of Eugene, Oreg.). Other labels include dyes, enzymes, other chemiluminescent molecules, particles (e.g., colloidal particles), radioisotopes (e.g., ³H, ³⁵S, or ¹²⁵I), or other directly or indirectly detectable agents.

If not directly labeled, the complex can be used in conjunction with secondary labeled immunoreagents, which will specifically bind the complex. Instead of a labeled multivalent binding partner, a second stage label (e.g., an antibody that specifically binds the complex or a component thereof) may be used. Methods in which the multimeric complexes are detected with a single direct staining method or indirectly by fluorescence may be more convenient, but the complexes can also be detected using two-step staining protocols, such as the secondary antibodies referenced above. Indirect measurements can also be made where the complex includes substrates for enzymatic reactions that produce a colored or otherwise detectable product (e.g., substrates for horseradish peroxidase, alkaline phosphatase, malate dehydrogenase and the like).

Methods of making: The complexes described above, and subunits thereof (e.g., subunits that contain a receptor-binding molecule and a linker) can be made in a variety of ways. For example, using standard recombinant techniques, one can prepare a sequence (e.g., a cDNA sequence) that encodes any given receptor-binding molecule (or a receptor-binding fragment or other mutant thereof, e.g., a chemokine or receptor-binding fragment thereof) and any given linker (e.g., a BSP; Schatz, Biotechnol.11:1138-1143, 1993), and clone that sequence into a suitable expression vector (e.g., a pET vector, which is one in a series encompassed by the pET System (Novagen, Darmstadt, Germany)). The pET vectors were developed for the cloning and expression of recombinant proteins in E. coli. In these vectors, target genes are cloned under control of strong bacteriophage T7 transcription and translation signals, and expression is induced by providing a source of T7 RNA polymerase in the host cell.

Cloning may be facilitated by reverse transcription polymerase chain reaction (RT-PCR), and/or by subcloning from an intermediate vector construct. The sequences can be cloned adjacent to one another (i.e., the sequences can be contiguous; they may, however, also be separated by one or more additional amino acid residues) so that a fusion protein (e.g., a chemokine-BSP fusion) is produced upon expression.

The encoded sequences can be placed under the control of an inducible or constitutively active promoter (or any sequence sufficient to direct transcription), many of which are known in the art. For example, as with the pET vectors, the promoter can be the T7 bacterial promoter. Thus, expression can be driven not only by a native transcriptional initiation region, but also from an exogenous transcriptional initiation region (i.e., a promoter other than the promoter that is associated with the gene in a naturally occurring chromosome). The promoter can be introduced by recombinant methods in vitro, or as the result of homologous integration of the sequence into a chromosome. A wide variety of transcriptional initiation regions are known for a wide variety of expression hosts, which may be prokaryotes or eukaryotes (e.g., E. coli, B. subtilis, mammalian cells such as CHO cells, COS cells, monkey kidney cells, lymphoid cells, and the like). Generally, a selectable marker that is operative in the expression host will be present.

One can generate expression cassettes that include a transcription initiation region, a sequence encoding a receptor-binding molecule, and a transcriptional termination region, the gene encoding the receptor-binding molecule and a transcriptional termination region optionally having a signal for attachment to a polyA sequence. Suitable restriction sites can be engineered into the terminus of the sequence encoding the receptor-binding molecule for cloning purposes. Sequences in the expression vector, whether within coding or noncoding regions, can be engineered by various means (e.g., they can be introduced during a polymerase chain reaction, by site directed mutagenesis, etc.). Accordingly, these techniques can be used to generate fragments or receptor-binding mutants of the receptor-binding molecules.

Standard methodologies (e.g., electroporation, biolistics, or calcium-phosphate precipitation) can then be used to introduce the expression vector into a cell (e.g., an insect, mammalian or bacterial cell (e.g., E. coli)) in which the fusion protein (receptor-binding molecule-linker) can be expressed. Of course, plasmids are not the only suitable expression vector. Cosmids, YACs, BACs, and viral vectors (e.g., retroviral vectors) can also be used. Different vectors may have properties particularly appropriate to give protein expression in the recipient. Likewise, the properties may differ for purposes of cloning, and a vector may also have different selectable markers.

If necessary, the expressed receptor-binding molecule, whether or not fused to a linker, can be solubilized. Conditions that permit resolubilization and protein folding are known in the art (see Current Protocols in Protein Science, John Wiley and Sons, 2002; Vuillard et al., Eur. J. Biochem. 256:128-135, 1998).

Regardless of the expression vector, once expressed, the protein can be purified (here again, suitable techniques for sufficient protein purification are known in the art) and, if necessary, modified further. The modification can take place at any time before the complex is formed. For example, if the expressed protein is meant to form a complex with avidin or streptavidin, the linker can be biotinylated; if the expressed protein is meant to form a complex with a multivalent binding partner that recognizes (and specifically binds or otherwise associates with) glycosylated, farnesylated, or phosphorylated proteins, the linker can be glycosylated, farnesylated, or phosphorylated. Once provided, this portion of the complex can be combined with an appropriate multivalent binding partner. For example, fusion proteins modified to contain a biotinylated linker can be combined with avidin or streptavidin.

The group introduced by the modifying enzyme (e.g., biotin, a sugar, a phosphate group, farnesyl, etc.) provides a complementary binding pair member or a site that, upon further modification (e.g., biotinylation, etc.) provides a complementary binding pair member. An alternative strategy is to introduce an unpaired cysteine residue to the monomer (i.e., the portion of the complex that includes the receptor-binding molecule), thereby introducing a unique and chemically reactive site for binding. The attachment site may also be a naturally occurring or introduced epitope, where the binding partner will be an antibody (e.g., an IgG or IgM antibody). Preferably, modifications occur at a site (e.g., a C-terminus) that will not substantially interfere with receptor binding.

For example, one can introduce the recognition sequence for the enzyme BirA, which catalyzes biotinylation of the protein substrate. The monomer (e.g., receptor-binding molecule) with a biotinylated subunit is then bound to a multivalent binding partner (e.g., avidin or streptavidin). Avidin and streptavidin have a valency of four, providing a complex with four receptor-binding molecules (e.g., a chemotetramer).

The multivalent binding partner may be free in solution, or may be attached to an insoluble support (any composition to which the complex binds; insoluble supports are readily separated from soluble material, and they are otherwise compatible with the reagents to which they are exposed in the process of detecting or isolating receptor-bearing cells). The surface of the support can be solid or porous, and the support can have any convenient shape. Examples of suitable insoluble supports include beads (e.g., magnetic beads), membranes (e.g., nylon, nitrocellulose, or other membranes) and microtiter plates (e.g., glass or plastic plates (e.g., polypropylene or polystyrene).

The multivalent binding partner can be made by the same recombinant techniques used to produce the fusion protein, or they may be purchased from commercial suppliers. Similarly, although more laborious, the components included in the fusion protein, the complex, or in the methods to make either, can be synthesized (e.g., on a protein synthesizer) or purified from biological tissues that contain them.

The individual components of the complex and the multimeric complex are both preferably stable over reasonably long periods of time (although unstable complexes are within the scope of the invention as well; no particular degree of stability is required, but rather preferred for ease of use). For example, preferably not more than about 10% (e.g., 5, 10, or 15%) of the multimeric complexes will dissociate when stored at 4° C. for about one day, and they may remain stable for more than about one week.

In addition to the methods in which complexes are assembled by virtue of the affinity of their components for one another (e.g., the affinity of avidin or streptavidin for a biotinylated protein), the complex can also be formed by chemically cross-linking monomeric proteins. A number of agents capable of cross-linking proteins are known in the art, including azidobenzoyl hydrazide, N-[4-(p-azidosalicylamino)butyl]-3′-[2′-pyridyldithio]propionamide, bis-sulfo-succinimidyl suberate, dimethyladipimidate, disuccinimidyltartrate, N-γ-maleimido-butyryloxysuccinimide ester, N-hydroxysulfosuccinimidyl-4-azidobenzoate, N-succinimidyl[4-azidophenyl]-1,3′-dithiopropionate, N-succinimidyl[4-iodoacetyl]aminobenzoate, glutaraldehyde, formaldehyde, 3,3′-Dithiobis[sulfosuccinimidylpropionate] (DTSSP), and succinimidyl 4-[N-maleimidomethyl]cyclohexane-1-carboxylate. These cross-linking agents and others like them can be used to form the complexes of the present invention.

Consistent with our discussion above, the crosslinking agent can be selected based on the strength of the association desired. For example, a cross-linker that functions by creating disulfide bonds (e.g., DTSSP) between members of the complex can be used to generate a complex that will disassemble upon uptake into a cell; upon internalization, the bonds are reduced, thereby releasing a cargo, such as a toxin or nucleic acid, that is attached to the complex. The strategies for complex disassembly that are used for synthesis of immunotoxins can be used in the context of the present invention (see, for example, Thorpe and Ross, Immunol. Rev. 62:119-158, 1982; Thorpe et al., Cancer Res. 47:5924-31, 1987; and Myers et al., Immunol. Methods 136:221-37, 1991).

To vary the ratio of the various members of the complex (i.e., the ratio of receptor-binding molecules to multivalent binding partners), one can vary the amount of each member present in solution. For example, if one desires a complex containing four receptor-binding molecules per multivalent binding partner (presuming the multivalent binding partner can accommodate four monomers), the components can be combined in a molar ratio of 4:1. In some circumstances, such as when the binding affinity of a linker molecule to the multivalent binding is weaker, it may be desirable to combine the components in a 5:1, 6:1, 10:1 ratio, or higher, to drive complex formation. It is not necessary to fill all the valency sites for a multivalent binding partner before using the multimeric binding complexes of the invention. A population of the multimeric binding complexes of the invention will work effectively if a mixed number of valencies are occupied on each molecule. For example, a streptavidin molecule that is bound by 1, 2, 3, or 4 biotin-CCL19 chemokine molecules can bind a complex with a CCR7 receptor. Therefore, a sample that contains a mixed population of the streptavidin molecules associated with 1, 2, 3, or 4 biotin-CCL19 molecules can be used to label cells expressing a CCR7 receptor.

At some time during the preparation of the complex, receptor-binding molecules that are not properly fused to linkers or receptor-binding molecule-linker fusion proteins that do not participate in complex formation may be removed (i.e., separated) from a mixture that contains them.

Methods of Detecting Receptors or Analogs Thereof: The complexes described herein can be used to detect receptors (a term used herein to refer to anything that is specifically bound by a receptor-binding molecule (or “ligand”); a “receptor” may be a single cell or membrane-associated protein, a complex of such proteins (e.g., the receptor complex that binds IL-2 or the receptor complex that binds IL-15), or another ligand-binding agent (e.g., a purified or synthesized protein or other chemical entity that binds ligands; a receptor analog)). While the receptor can have a naturally occurring sequence, it can also be a biologically active mutant (i.e., a mutant that retains the ability to bind a ligand). Receptors can be detected on cells in vivo or on cells, cell fractions, or membrane preparations ex vivo (e.g., in cell culture); the cells can be obtained from a patient or they may be obtained from commercially available sources of cell lines; the cells can express a given receptor naturally, or they can be genetically engineered to express a receptor or combination of receptors; and any of these cells can be placed in, for example, a suspension or solution for testing. As noted elsewhere, the cells can be of any type, so long as they are suspected of expressing a particular receptor (e.g., T cells can be tested to determine the number or identity of their chemokine receptors).

To detect a receptor, a complex of the invention can be (but is not necessarily) attached to a support structure (e.g., a solid or insoluble support), such as a bead (e.g., a magnetic bead), a membrane, or a microtiter plate. The support structure can be, or can be coated with, glass, plastic (e.g., polystyrene or polypropylene), nylon, or nitrocellulose. Receptors, whether free or membrane-bound (e.g., cell-associated), are then applied to the support, which is subsequently washed to remove unbound material. Receptors attached to the support can then be detected with, for example, one or more antibodies. For example, cells bearing receptors can be identified with cell-type specific antibodies. The immobilized complex can be any of those described herein, including chemotetramers.

Conversely, and perhaps more likely, the receptor (or a membrane or cell with which it is associated) will be immobilized and then exposed to one or more types of complexes; unbound complexes would then be washed away and the signal generated by the complex used to detect the receptor. In this way, one can detect and characterize receptors (whether naturally occurring or analogs) having an ability to bind the ligand within the tested complex.

A receptor (or a membrane or cell with which it is associated) can also be detected in vivo or in solution; it is not necessary to immobilize it on a solid support. All that is required is exposure to a suitable complex and a means to separate receptor-bound complexes from unbound complexes. The conditions under which the exposure takes place can vary. For example, a multimeric binding complex can be added to a suspension of the cells of interest, and incubated for a time and at a temperature that will permit binding to occur if the cell expresses a receptor for the receptor-binding portion of the complex. One of ordinary skill in the art is well able to determine acceptable incubation conditions, which can vary from about 4° C. to room temperature (about 25° C.) to body temperature (about 37° C.) for several minutes (e.g., 5 to 30 minutes) to several hours (e.g., 2-24 hours). The medium can be any suitable medium as known in the art. If live cells are desired, a medium will be chosen that maintains the viability of the cells. A preferred medium is phosphate buffered saline (PBS) containing from about 0.1 to 0.5% BSA. Various media are commercially available and may be used according to the nature of the cells, including Dulbecco's Modified Eagle Medium (DMEM), Hank's Basic Salt Solution (HBSS), Dulbecco's phosphate buffered saline (DPBS), RPMI, Iscove's medium, PBS with 5 mM EDTA, etc. These media are frequently supplemented with fetal calf serum, BSA, HSA, etc. and may also contain an antibiotic.

A number of methods for detecting and quantitating labeled cells are known in the art, and these methods can be used to detect and quantitate cells bound by the complexes described herein. Flow cytometry is a convenient way to detect labeled cells that constitute a small percent of the total population, and it can be used to separate labeled cells from a complex mixture of cells. The cells may be collected in any appropriate medium (e.g., a medium that maintains the viability of the cells; various media are commercially available). The collection tube may contain a cushion of serum at the bottom. Fluorescent microscopy may also be used. Various immunoassays (e.g., enzyme-linked immunosorbent assays (ELISAs), radioimmunoassays (RIAs), etc.), may be used to quantitate the number of cells bound to an insoluble support. The complex can be bound to the support by any convenient means. After incubation, the insoluble support is washed to remove non-bound components. Several washes (e.g. from one to about six washes) can be carried out with a volume sufficient to thoroughly wash away non-specifically bound cells. The receptor-bearing cells are then eluted from the binding complex. In particular, the use of magnetic particles to separate cell subsets from complex mixtures is described in Miltenyi et al. (Cytometry 11:231-238, 1990).

The assays can be described as “sandwich” assays where the multimeric binding complex is attached to an insoluble surface or support. Attachment can be by any convenient means, depending on the nature of the surface and can be made either directly or through specific antibodies.

Before adding a sample (e.g., a patient sample, cultured cells, receptors or receptor analogs), any non-specific binding sites on the insoluble support (i.e., those not occupied by the multimeric binding complex) are generally blocked. Preferred blocking agents include non-interfering proteins such as bovine serum albumin (BSA), casein, gelatin, and the like. Samples, fractions or aliquots thereof are then added to separately assayable supports (for example, separate wells of a microtiter plate) containing support-bound multimeric binding complex.

In certain settings (e.g., clinical settings) it is convenient to perform the assays in a self-contained apparatus, and a number of these are known in the art. Generally, they employ a continuous flow-path of a suitable filter or membrane, having at least three regions: a fluid transport region, a sample region, and a measuring region. The sample region is prevented from fluid transfer contact with the other portions of the flow path prior to receiving the sample. After the sample region receives the sample, it is brought into fluid transfer relationship with the other regions, and the fluid transfer region contacted with fluid to permit a reagent solution to pass through the sample region and into the measuring region. The complex can be bound to the measuring region.

Detecting particular receptor-bearing cells is useful in diagnosing or monitoring conditions in which those cells are affected. For example, detecting T cells is useful in connection with a variety of conditions associated with T cell activation. Such conditions include autoimmune diseases (e.g., multiple sclerosis, myasthenia gravis, rheumatoid arthritis, type I diabetes, graft vs. host disease, Grave's disease, etc.) and various forms of cancer (e.g., carcinomas, melanomas, sarcomas, lymphomas and leukemias). Various infectious diseases are also of interest, including those caused by viruses (e.g., human immunodeficiency viruses (HIV), a hepatitis virus, a herpesvirus, an enteric virus, a respiratory virus, a rhabdovirus, a rubeola virus, a poxvirus, a paramyxovirus, a morbillivirus, etc. . . . . Infectious diseases can also be caused by non-viral agents, including bacteria, such as Pneumococcus sp., Staphylococcus sp., Bacillus sp., Streptococcus sp., Meningococcus sp., Gonococcus sp., Eschericia sp., Klebsiella sp., Proteus sp., Pseudomonas sp., Salmonella sp., Shigella sp., Hemophilus sp., Yersinia sp., Listeria sp., Corynebacterium sp., Vibrio sp., Clostridia sp., Chlamydia sp., Mycobacterium sp, Helicobacter sp. and Treponema sp., protozoan pathogens, and the like. T cell-associated allergic responses may also be monitored (e.g., delayed type hypersensitivity or contact hypersensitivity involving T cells).

There are many other conditions in which it will be useful to determine or monitor the number or type of certain receptor-bearing cells. For example, one can detect and/or monitor cells having specific chemokine receptors. Cellular chemokine receptors facilitate certain infectious diseases because they are exploited as cell entry and disease transmission factors by intracellular pathogens (reviewed in Murphy et al., 2000). For instance, CCR5 is a co-receptor for HIV and is implicated in the progression of AIDS, and the Duffy Antigen Receptor is known to play a role in the progression of the form of malaria caused by Plasmodium vivax. The CXCR4 receptor and others are also known to function as HIV co-receptors, but their role in the disease is not entirely understood (Horuk et al., Immunol. Today 15:169-174, 1994; Rucker et al., J. Virol. 71:8999-9007, 1997; and Berger et al., Ann. Rev. Immunol. 17:657-700, 1999). By other mechanisms, herpesvirus- and poxvirus-encoded chemokines and chemokine receptors, apparently acquired as copied host genes, may subvert the immune response or dysregulate cell growth (reviewed in Pease and Murphy, Semin. Immunol. 10:169-178, 1998). The use of complexes that include chemokines (or fragments or mutants thereof) can be used to expand an understanding of chemokines and are useful in developing therapeutic agents for chemokine-receptor mediated disease. The complexes themselves can be used to treat or prevent (i.e., to confer a therapeutic benefit on a patient, whether evident as an objective or subjective improvement in the patient's health) diseases or conditions associated with chemokine receptor binding.

Quantitative assays: The complexes described herein can be used to distinguish populations of cells having a particular receptor or a particular constellation of receptors. In some cases, it is beneficial to determine the number of cells or receptors in a population of cells (e.g., a biological sample) with some precision. This information can be used to determine whether a population of cells in an experimental sample is different from the population of cells in a control sample. For example, one can determine whether the number of cells in an experimental sample has increased or decreased (or come to express more or fewer receptors) relative to a control sample obtained at an earlier time (e.g., before a patient was exposed to an infectious agent or before a treatment regime began). Similarly, one can determine whether the number of cells in an experimental sample (e.g., an organ or tissue affected by an infectious agent) has increased or decreased (or come to express more or fewer receptors) relative to a control sample obtained from a different organ or patient (e.g., an organ or patient unaffected by an infectious agent). The cells can then be sorted into populations that contain or express low levels of a given receptor (e.g., the CCR7 receptor, the NGF receptor, the TNF receptor, or the like) from cells that contain or express higher levels of the same receptor.

The cells examined can be from essentially any biological sample or they may be cells that have been maintained in culture for a certain period of time. For example, the sample can be a blood or lymph sample. Other samples of interest are tissues, or associated fluids, such as the brain, cerebrospinal fluid, or other parts of the nervous system, lymph nodes, a neoplasm or other suspicious tissue growth, spleen, liver, kidney, pancreas, tonsil, thymus, tissue from a joint or synovial fluid, and the like. The sample may be used essentially as obtained (particular where the sample is liquid) or it can be modified (e.g., the cells can be dissociated and then either diluted (e.g., diluted 1:10 and usually not more than about 1:10,000) or concentrated). The sample can also be treated by centrifugation, Ficoll-Hypaque, panning, affinity separation (e.g., using antibodies specific for one or more markers present as surface membrane proteins), or any other technique that enriches the set or subset of cells of interest.

Some ligands (e.g., some chemokines) may bind more than one receptor, and that knowledge can be used in designing the complexes of the invention. For example, to identify cells that express receptors that bind a promiscuous chemokine, the least promiscuous of the possible chemokines can be selected. For example, to identify cells expressing CCR1, CCR5 and D6 receptors, MIP-1α and RANTES binding complexes can be utilized.

As noted above, it may be desirable to quantitate the number of cells that express a particular receptor or receptor complex, although determining the exact number of cells present is not always necessary. In some instances, a rough or roughly accurate estimate may be sufficient. Similarly, it may only be important to detect a change (e.g., an increase or decrease in cell number or receptor expression) from a prior level. For example, quantitative assays can be performed to monitor the progression of a number of conditions associated with T cell activation, including autoimmune diseases, graft rejection, or infection (e.g., viral, bacterial, or protozoan infection).

A great many cell types (essentially any of the cells that respond to secreted signaling molecules) can be analyzed with a receptor-binding complex having the essential features described herein. For example, one can analyze the leukocytes referred to above, which respond to chemotactic signals mediated by cytokines.

Cells (e.g., T cells, including resting, effector or memory T cells) that express a particular receptor can be separated from complex mixtures, such as biological samples, once they are bound to a complex. In this way, any cell-bearing sample can be enriched or depleted in particular cell types (i.e., those expressing a receptor bound by a component of the complex).

Arrays: Methods for attaching complexes to solid supports have been described above, and other such techniques are known to those of ordinary skill in the art. Microtiter plates are especially convenient because a large number of assays can be carried out simultaneously using small amounts of reagents and samples. Microtiter plates or, alternatively, complexes arrayed on another solid support (e.g., glass or a membrane) can be used to identify a complement of receptors expressed on a given cell population. Accordingly, arrays of complexes and methods of using them to detect, separate, quantitate, or otherwise characterize receptor molecules (e.g., cell-bound receptors as well as receptor analogs) are within the scope of the invention. For example, the invention features a solid support (e.g., one of those described above) upon which two or more (e.g., 2, 5, 8, 10, or 12) complexes are distinctly positioned (i.e., are placed at identifiable and essentially non-overlapping positions). The arrays can contain many distinctly positioned complexes (e.g., 5, 8, 10, or 12 complexes or multiples thereof (e.g., 15, 20, 25, 30, 35, 40, 45, 50, etc complexes); large arrays can contain hundreds or even thousands of complexes). The arrays can be used to identify cells that express receptors that bind any of the receptor-binding molecules of the arrayed complexes. In this way, various populations of cells can be characterized; a homogeneous or heterogeneous population of cells that bind complexes at, for example, 18 of 50 positions on the array, express receptors for the ligands (or receptor-binding molecules) at those 18 positions, but do not express receptors for the ligands (i.e., receptor-binding molecules) at the remaining 32 positions. Cells can be characterized at any point in their development; before or after a treatment regime; or when obtained from healthy vs. diseased tissue. Moreover, the arrays can contain complexes that include a variety of types of receptor-binding molecules or receptor-binding molecules of the same type. For example, the array can contain complexes that include only hormones as the receptor-binding molecule; that include only neurotransmitters as the receptor-binding molecule; that include only chemokines as the receptor-binding molecules; etc.

As noted above, the complexes of the invention, optionally arrayed (or, alternatively, exposed to an array), can also be used to identify receptor analogs or receptor agonists or antagonists. For example, to identify a receptor analog, one can array potential analogs and expose them to a complex bearing a known receptor-binding molecule (for example, if searching for an analog of the NGF receptor, one could array potential analogs and expose the array to complexes that contain, as the receptor-binding molecule, NGF). Potential analogs bound by the complex can then be identified by virtue of a detectable label (e.g., a detectable label carried by the complex). Alternatively, arrays of complexes can be exposed to compound libraries (e.g., collections of small molecules or polypeptides (e.g., variously mutated polypeptides)); the substances that bind particular complexes may serve as analogs (or mimics) of a given receptor. Of course, this assay can be carried out with a single type of complex as well (an array is not required).

Methods of using the complex to deliver moieties to cells: The specificity of the interaction between the complexes described herein and biological cells can be exploited to deliver moieties (e.g., nucleic acids (e.g., DNA, or single- or double-stranded RNA), other therapeutic molecules, or cytotoxins) to those cells. Prior to delivery, one can identify or obtain a cell that would benefit from exposure to a therapeutic agent or cytotoxin. While in many instances therapeutic agents and cytotoxins are distinct, there are cases in which a single moiety could be described as either (or both). For example, a patient who has cancer has cells that would benefit from exposure to a chemotherapeutic agent that kills the cancerous cell. In other instances, the therapeutic agent will not be cytotoxic. To the contrary, it can be a nucleic acid molecule that encodes a protein that is defective or deficient in the cell; that inhibits the expression of a protein that is defective or overproduced in the cell; or that is expressed by a pathogen within the patient or the patient's cells (e.g., a bacterial, viral, or parasitic agent). Any cell presently known to be a target for genetic therapy can be targeted using the complexes of the invention; all that is required is to identify a receptor specifically expressed by the target cells and to attach the therapeutic moiety (e.g., a nucleic acid sequence encoding a defective protein) to a complex containing a ligand (receptor-binding molecule) that binds (and, preferably, specifically binds) a receptor identified on the target cell.

While the complexes of the invention can include known therapeutic agents, complexes that include only a multivalent binding partner, a receptor-binding molecule and, optionally, a linker can be administered to treat a wide variety of diseases or conditions. These include but are not limited to, cancer, autoimmune diseases (e.g., rheumatoid arthritis, multiple sclerosis, diabetes, and systemic lupus erythematosis), neurological diseases (including mental illnesses such as depression) and growth-associated disorders (e.g., dwarfism). For example, complexes in which the receptor-binding molecule is a growth factor, such as transforming growth factor-betal or fibroblast growth factor can be administered to facilitate tissue (e.g., bone or skin) regeneration (Walsh et al., Cell Tissue Res 311:187-98, 2003). Such complexes may be particularly useful in treating chronic wounds. Complexes containing fibroblast growth factor-2 can be used to treat atherosclerosis (Rinsch et al., Gene Ther 8:523-33, 2001). In other embodiments, complexes containing cytokines in the interleukin (IL)-6 family can be used to treat patients who have multiple sclerosis (MS) (Ransohoff et al., Trends Immunol. 23:512-6).

The nucleic acid, cytotoxin, or other therapeutic agent (i.e., a “cargo”) can be attached to at least one of the receptor-binding molecules within a multimeric complex that will specifically recognize the identified receptor, thus creating an even larger complex. Alternatively, the “cargo” can be attached to the multivalent binding partner or the linker molecule, such as on a biotin linker. Any of the complexes described herein can further include a nucleic acid, cytotoxin, or other therapeutic agent. Complexes bearing such agents and cells that contain them are also within the scope of the invention.

The expanded complex can be brought into contact with a target cell in a variety of ways. For example, the two can be incubated together in culture or in solution. Alternatively, the complex can be administered to a patient (routes of administration are described below) or applied to a patient's tissue (in vivo or following explantation (e.g., prior to re-implantation or transplantation)). While the invention is not limited to compositions that act by any particular mechanism, or to methods in which any particular event occurs, it is thought that complexes containing a therapeutic moiety are taken into the cell by endocytosis. Depending on the cargo, dissociation of the cargo and the multimeric complex is not always necessary to elicit a desired effect on a cell. For example, some cytotoxins can induce cell death even when the toxin is still attached to the multimeric complex. In another strategy, the cargo can be attached to the receptor-binding molecule by a disulfide linkage. Upon internalization of the complex, the disulfide bond is reduced, and the cargo is released from the complex. This strategy is commonly used for the development of immunotoxins (see, for example, Thorpe and Ross, Immunol. Rev. 62:119-158, 1982; Thorpe et al., Cancer Res. 47:5924-31, 1987; and Myers et al., Immunol. Methods 136:221-37, 1991). Another strategy to facilitate dissociation of the cargo from the complex is to use a linker/multimeric binding complex combination that has a higher dissociation constant (K_{D}; i.e., lower binding affinity) than other combinations. For example, as discussed supra, the Strep-Tag II binds Strep-Tactin with an affinity that is 100 times greater than its affinity for streptavidin (IBA, St. Louis, Mo.). The cargo can be fused to Strep-Tag II and then complexed with streptavidin. The faster off-rate will facilitate release of the cargo from the complex. A similar strategy can be employed in designing methods to deliver a cargo to a cell by attachment of the cargo to the receptor molecule instead of to the multivalent binding partner. In some cases it may be preferable to couple the cargo to the multivalent binding partner (rather than to the receptor molecule) to avoid interference of the cargo with receptor-binding molecule/receptor interactions.

In the event the therapeutic moiety is an RNA molecule, the RNA molecule can be single stranded (e.g., an antisense RNA or a ribozyme) or double stranded (e.g., a small inhibitory RNA (siRNA)). Antisense RNA molecules are typically the reverse and complement of a targeted mRNA sequence; antisense RNA hybridizes with and subsequently inactivates mRNA. Ribozymes are structured RNAs that catalyze the cleavage of target RNAs. The siRNA is typically a short (e.g., 21-23 nucleotides) double stranded RNA containing 1-2 nucleotide 3′ overhangs. One strand of the dsRNA is identical to, or homologous to, the target RNA. The siRNA directs target RNA cleavage by the RNAseIII-like enzyme Dicer within the RNA induced silencing complex (RISC).

When a given complex is brought into contact with a target cell (by, for example, incubating the complex in solution with the cell or by placing the complex in contact with a tissue cell culture or in vivo), the receptor on the target cell will bind the cognate ligand (i.e., the receptor-binding molecule) and the cell will subsequently take up the complex by endocytosis.

As noted, the complexes can also be used to deliver cytotoxins, which can include cytotoxic antibodies, polysaccharides or chemical compounds (e.g., small molecules), to cells expressing defined receptors. Genes encoding proteins that trigger apoptosis are also cytotoxic agents. Specific cytotoxins, any of which can be incorporated in the complexes of the invention, include ricin, abrin, diptheria toxin, maytansinoids, cisplatin, and the like. Where there are two subunits, only the cytotoxic subunit may be used (e.g., the α-unit of ricin). The toxin (or other agent slated for delivery) can be conjugated to the binding complex in a variety of ways. For example, it can be joined by means of a cross-linker or by way of a disulfide bond. Toxin conjugates are disclosed in, for example, U.S. Pat. Nos. 5,208,020; 4,863,726; 4,916,213; and 5,165,923.

Formulations and Routes of Administration: Pharmaceutical compositions for use in accordance with the present invention may be formulated in a conventional manner using one or more physiologically acceptable carriers or excipients. Thus, the multimeric complexes may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration the compositions may take the form of tablets or lozenges formulated in conventional manner.

For administration by inhalation, the complexes for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the complex and a suitable powder base such as lactose or starch.

The complexes may be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

The complexes may also be formulated in rectal compositions such as suppositories or retention enemas, for example, containing conventional suppository bases such as cocoa butter or other glycerides.

In addition to the formulations described previously, the complexes may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

The therapeutic compositions of the invention can also contain a carrier or excipient, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. The nucleic acids, polypeptides, antibodies, or modulatory compounds of the invention can be administered by any standard route of administration. For example, administration can be parenteral, intravenous, subcutaneous, intramuscular, intracranial, intraorbital, opthalmic, intraventricular, intracapsular, intraspinal, intracistemal, intraperitoneal, transmucosal, or oral. The modulatory compound can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences.”

It is recognized that the pharmaceutical compositions and methods described herein can be used independently or in combination with one another. That is, subjects can be administered one or more of the pharmaceutical compositions, for example, pharmaceutical compositions comprising a nucleic acid molecule or protein of the invention or a modulator thereof, subjected to one or more of the therapeutic methods described herein, or both, in temporally overlapping or non-overlapping regimens. When therapies overlap temporally, the therapies may generally occur in any order and can be simultaneous (e.g., administered simultaneously together in a composite composition or simultaneously but as separate compositions) or interspersed. By way of example, a subject afflicted with a disorder described herein can be simultaneously or sequentially administered both a cytotoxic agent which selectively kills aberrant cells and an antibody (e.g., an antibody of the invention) which can, in one embodiment, be conjugated or linked with a therapeutic agent, a cytotoxic agent, an imaging agent, or the like.

Effective Dose: Toxicity and therapeutic efficacy of the complexes disclosed in the invention (e.g., multimeric complexes containing cytotoxins; therapeutic molecules, such as antisense or double stranded RNA; or labeling molecules), and the compounds that modulate their expression or activity can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Polypeptides or other compounds that exhibit large therapeutic indices are preferred. While complexes that exhibit toxic side effects may be used, care should be taken to design a delivery system that targets such complexes to the site of affected tissue to minimize potential damage to uninfected cells and, thereby, reduce side effects.

Data obtained from the cell culture assays and further animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Levels in plasma may be measured, for example, by high performance liquid chromatography.

Kits: Additional compositions of the invention are kits that include one or more of the compositions described above (e.g., one or more of the multimeric complexes described herein or one or more of the components thereof (e.g., a receptor-binding molecule, optionally joined to a linker, which may or may not be modified to facilitate interaction with a multivalent binding partner). Alternatively, or in addition, a kit can include: (1) a vector (e.g., a cloning or expression vector for expressing any receptor-binding molecule, which can be fused to a protein or peptide containing a sequence that is recognized by a modifying enzyme (e.g., a BSP, which is recognized and modified by BirA) or any other component of a complex); (2) DNA sequences that can be expressed in the vector (e.g., any one or more of the receptor-binding molecules described herein, or any one or more of the linkers or multivalent binding partners described herein); (3) cells (e.g., bacterial cells or insect cells) that can be used to express the components of the complex (in some instances the cells may already be transfected with, and may stably express, one or more of the components of the complexes described herein); (4) reagents useful in purifying the expressed proteins (e.g., a chemokine-linker) from a cell; (5) a multivalent binding partner (e.g., avidin or streptavidin, optionally conjugated to a fluorescent or other detectable molecule); (6) a modifying enzyme (e.g., BirA, a glycosylase, a farnesyl protein transferase, a protein kinase, or the like); and (7) reagents for assembling a complex (e.g., reagents for binding a monomer to the multivalent binding partner). In addition, the kits can include instructions for assembling the complex and/or using it to detect the presence of a receptor (e.g., a receptor on the surface of a cell).

Kits helpful in assembling screening or selection assays can include one or more complexes bound to an insoluble support (e.g., a bead, such as a magnetic bead; a microtiter plates; a glass slide; and the like). Reagents used in the process of binding, washing, or detecting receptors can also be included. Where cell sorting is desired, kits can include reagents appropriate for use in the sorting process (e.g., in the process of flow cytometry).

Any of the kits described above can also include a buffering agent, a preservative, a protein-stabilizing agent, or a component necessary for detecting any included label (e.g., an enzyme or substrate). The kits can also contain a control sample or a series of control samples that can be assayed and compared to the test sample. Each component of the kit can be enclosed within an individual container, and all of the various containers can be within a single package.

The materials, methods, and examples described here and below are illustrative only; they are not intended to limit the scope of the invention, which is defined by the claims. The contents of all references, pending patent applications and published patents cited throughout our written description are hereby expressly incorporated by reference.

### EXAMPLES

### Example 1

### Methods

Donors and Samples: Samples were obtained from 12 healthy individuals. The blood samples were provided in either heparin- or EDTA-anticoagulant tubes. Peripheral blood mononuclear cells (PBMCs) were isolated from the blood samples over lymphocyte separation medium (Cellgro, Hemdon, Va.). In a few cases, cryopreserved PBMCs were used.

MHC class I/peptide tetramers: Soluble MHC class I/peptide tetramers carrying CTL-epitopes of CMV, EBV and flu proteins were produced as described elsewhere (Altman et al., Science 274:94-96, 1996). The HLA restriction, peptide sequences, the virus and the name of the gene products of derived CTL-epitopes are presented in Table 1. The tetramers were prepared with streptavidin coupled to either allophycocyanin (APC) or phycoerythrin (PE) (Molecular Probes, Eugene, Oreg.).

TABLE 1
MHC class I/peptide tetramers and HLA restricted epitopes.

| MHC/peptide | HLA | | Viral | |
|---|---|---|---|---|
| tetramers | restrictions | CTL epitope | infection | Viral protein |
| A2/CMV | A 0201 | NLVPMVATV | CMV | pp65 |
| A2/flu | A 0201 | GILGFVFTL | flu | MP |
| A2/EBV.GLC | A 0201 | GLCTLVAML | EBV | BMFL1 |
| B8/EBV.FLR | B 0801 | FLRGRAYGL | EBV | EBNA3A |
| B8/EBV.RAK | B 0801 | RAKFKQLL | EBV | BZLF1 |

Construction of pET-CCL19/BSP41 plasmid and expression of CCL19 monomer: The cDNA sequence for mature human CCL19-chemokine was produced by PCR amplification with primers CCL19.5P (5′-GGAATTCCATATGGGAACAAATGATGCTGAAGACTGC-3′ (SEQ ID NO:______)) and CCL19.3P (5′-CGGGATCCACTGCTGCGGCGCTTCAT-3′ (SEQ ID NO:______)), using the plasmid AI-pTyT30 (IncyteGenomics, St. Louis, Mo.) as a template. CCL19.5P contains a Nde I-restrictase adapter sequence, followed by the initiating ATG codon and the nucleotides complementary to the 5′ portion of the CCL19 coding sequence. CCL19.3P contains BamH I-restrictase adaptor sequence and nucleotides complementary to the 3′ portion of the CCL19 coding sequence preceding the TAA stop codon. The amplified DNA fragment was digested with Nde I and BamH I and inserted into the pJA1 plasmid linearized with the same enzymes. In this construct, the nucleotide coding sequence for mature CCL19 was placed downstream of the T7 promoter and upstream of the BSP41 biotinylation site coding sequence followed by a TGA-stop codon. The orientation of the insert and the authenticity of its sequence was verified by nucleotide sequence analysis. The resulting plasmid, pET-CCL19/BSP41, encodes a CCL19 protein fused with 17 amino acids of BSP41 biotinylation site in E. coli and its expression is regulated by the IPTG-inducible T7 promoter.

E. coli strain BL21(DE3) was transformed with pET-CCL19/BSP41 and analyzed for CCL19-monomer expression by SDS-PAGE on a small scale culture according to the pET System manual (Novagen, Darmstadt, Germany). The majority of the protein (65% of total sample) was expressed after 4 hours of IPTG induction in the form of inclusion bodies. The protein migrated as the major band at 11K. The pET series of plasmids nearly always produce proteins in inclusion bodies, and like all such systems, the inclusion body fraction is highly enriched for the protein of interest, but the protein is misfolded and must be solubilized in denaturant and refolded in vitro to produce native material. The chemokines are particularly amenable to this approach because they are small, stable and monomeric. The primary test for the integrity of the reagents is flow cytometry.

Preparation of CCL19-chemotetramer: A CCL19-monomer was prepared from 6 liters of cultured E. coli. The cells were collected by centrifugation after 4 hours of IPTG-induction and sonicated in 50 mL of resuspension buffer (50 mM Tris-HCl, pH 8.0; 25% sucrose; 1 mM EDTA, pH 8.0; 10 mM DTT; 1 mg/mL lysozyme; 5 mM MgCl₂; 0.4 mg/mL Dnase I). The insoluble material was collected by centrifugation at 20,000×g for 10 minutes and washed four times in wash buffer with Triton X-100 (50 mM Tris-HCl, pH 8.0; 0.5% Triton X-100; 2 M urea; 1 mM EDTA; 1 mM DTT) and once in wash buffer without Triton X-100. The insoluble material was collected by centrifugation and dissolved in extraction buffer (8 M Guanidine-HCl; 50 mM Tris-HCl, pH 7.4; 5 mM EDTA; 5 mM DTT). CCL19-monomer was purified by gel-filtration chromatography on a S100 column (Amersham-Pharmacia, Piscataway, N.J.) in gel-filtration buffer with guanidine-HCl (50 mM Tris-HCl, pH 7.5; 4 M guanidine-HCl; 2 mM DTT). The protein purity was more than 99%. The denatured protein (20 mg) was folded in 1 L of folding buffer (400 mM L-arginine; 100 mM Tris-HCl, pH 8.3; 2 mM EDTA; 5 mM reduced glutathione; 0.5 mM oxidized glutathione) at 10° C. for 72 hours. The folding reaction was concentrated to the volume of 7.5 mL by ultrafiltration, using a Biomax 5000 membrane in an Amicon 500 mL stir cell (Millipore Corporation, Bedford, Mass.) and exchanged against biotinylation buffer (100 mM Tris, pH 7.5; 200 mM NaCl; 5 mM MgCl₂) in a PD-10 column (Amersham-Pharmacia, Piscataway, N.J.). The folded CCL19-BSP41 (10.5 mL) in biotinylation buffer was mixed with 500 μl 100 mM ATP, pH 7.0; 40 μl 100 mM biotin; 10 μl mg/mL leupeptin (in H₂O); 10 μl 1 mM pepstatin (in methanol); 20 μl 100 mM PMSF and 10 μl BirA (20 μg) and incubated overnight at room temperature (RT). The biotinylated CCL19-monomer was purified by gel-filtration on a S100 column in gel-filtration buffer without Guanidine-HCl and DTT (50 mM Tris-HCl, pH 7.5; 150 mM NaCl). The appropriate fractions were collected, concentrated in an ultrafree-15 centrifuge filtering tube and dialyzed three times against 1 L of PBS without calcium and magnesium (Cellgro, Herndon, Va.). The CCL-19 monomer (10 μl) was incubated with a total of 32.4 μl of 2 mg/mL PE-Streptavidin or 18 μl of 2 mg/mL APC-Streptavidin (Molecular Probes, Eugene, Oreg.) for 1 hour and 40 minutes at RT.

Intracellular cytokine staining: PBMCs (1×10⁶ cells) were stimulated with an appropriate peptide (10 μg/mL) in 1 mL RPMI1640/10% FBS media, containing co-stimulatory antibodies CD28 (1 μg/mL) and CD49d (1 μg/mL) and GolgiPlug (Pharmingen) for 6 hours at 37° C. After peptide stimulation, intracellular cytokine staining was carried out using Cytofix/Cytoperm Plus kit (Pharmingen). Peptide stimulated PBMCs were stained with CD8-PerCP for 15 minutes at 4° C., and washed twice with 2 mL FACS buffer (PBS, 2% BSA). The cells were permeabilized with Cytofix/Cytoperm, washed twice with Perm/Wash buffer, stained with IFNγ-FITC, TNFα-PE and CD3-APC for 15 minutes at 4° C., washed again and fixed with 1% paraformaldehyde prior to acquisition of the data on a FACS Calibur (Becton Dickinson). FACS data were analyzed with FlowJo software (TreeStar, San Carlos, Calif.).

Immunotyping of lymphocytes: Cells were stained with FITC, PE, PerCP and APC labeled antibodies or tetramers, using standard methods. Any remaining free biotin binding sites in PE- and APC-labeled tetramers were quenched by the addition of biotin (1 mM), followed by a 30-minute incubation on ice prior to staining cells. The whole blood samples (200 μl) were stained at room temperature for 15 minutes; red blood cells were lysed in FACS lysing solution (Becton Dickinson, San Jose, Calif.) for 10 minutes, washed twice in FACS buffer and fixed in 200 μl of 1% paraformaldehyde. PBMCs (1×10⁶ cells/100 μl) were stained at 4° C. for 15 minutes, washed twice with 2 mL FACS buffer and fixed with paraformaldehyde. The following FITC-labeled antibodies (Beckman Coulter) were used: CD3, CD11a, CD16, CD27, CD28, CD38, CD45RA, CD56, CD62L and HLA-DR. CD8-PerCP and unlabeled CCR7 IgM were purchased from Becton Dickinson. The mAbs CD20-APC, CD3-APC were obtained from Beckman Coulter. Staining with unlabeled CCR7 antibody was detected by a secondary anti-mouse-FITC antibody (Jackson ImmunoResearch).

Generation of CCL19 chemotetramer: The CCL19 chemotetramer was engineered to generate a staining reagent for phenotypic characterization of Ag-specific CD8⁺ T cells. The cDNA sequence for mature CCL19 (Yoshida et al., J Biol. Chem. 272:13803-13809, 1997), lacking an N-terminal signal peptide sequence, was fused at the C-terminal end with a BSP-41 coding sequence. Expression in E. coli generated a CCL19 chemokine tagged with a BSP41 peptide (LHHILDAQKMVWNHR; Shatz, Biotechnology 11:1138-1143, 1993). The recombinant protein was expressed in E. coli using a T7 polymerase expression system. The protein was expressed in inclusion bodies, which were extensively washed prior to solubilization in guanidine hydrochloride. Denatured CCL19 was purified by gel filtration chromatography, folded, biotinylated and multimerized into a tetramer with either streptavidin-PE or -APC. The primary reason for using the chemokine in the tetrameric form was to avoid protocols that require indirect staining.

### Example 2

### CCL19 Chemotetramers Bound Subpopulations of Human PBMCs

FIGS. 1 and 2 show CCL19-chemokine staining on four major subsets of peripheral blood mononuclear cells (PBMC) in humans. No staining was observed on CD16⁺CD56⁺ lymphocytes, representing NK cells, and a moderate level of staining was visible on nearly all B cells, identified as CD20⁺. The populations of CD3⁺CD4⁺ and CD3⁺CD8⁺ exhibited the highest level of CCL19-chemotetramer staining, with more CD3⁺CD4⁺ than CD3⁺CD8⁺ cells that stain positive with the CCL19 tetramer. Within the CD3⁺CD8⁺ population, CCL19-chemotetramer positive cells could be divided into intermediate and high subsets.

Studies on migratory responses of human and mouse lymphocytes have demonstrated that NK cells are completely unresponsive to CCL19, while both B and T cells are capable of chemotaxis toward CCL19 (Campbell et al., J. Cell Biol. 141:1053-1059, 1998). Thus, the staining profile of CCL19-chemotetramer determined in the present study directly correlated with the CCR7 migratory responses of the cells examined.

### Example 3

### CCL19-Chemotetramer Bound Specifically to CCR7

Prior to staining of whole blood, the CCL19-chemotetramer was pre-incubated with increasing concentrations of anti-CCL19 mAb. The staining was examined by flow cytometry. Anti-CCL19 mAb (1 μg) completely blocks the CCL19-chemokine staining on CD3⁺CD8⁺ and CD3⁺CD8⁻ cells. The staining can also be blocked with heparin (FIG. 4), which is known to bind a number of chemokines (von Andrian and Mackay, N. Engl. J Med. 343:1020-1034, 2000). CCL19-chemotetramer staining was also blocked with unlabeled human CCL19-chemokine (FIG. 5). These results indicated that CCL19-chemotetramer bound to its target in a specific manner.

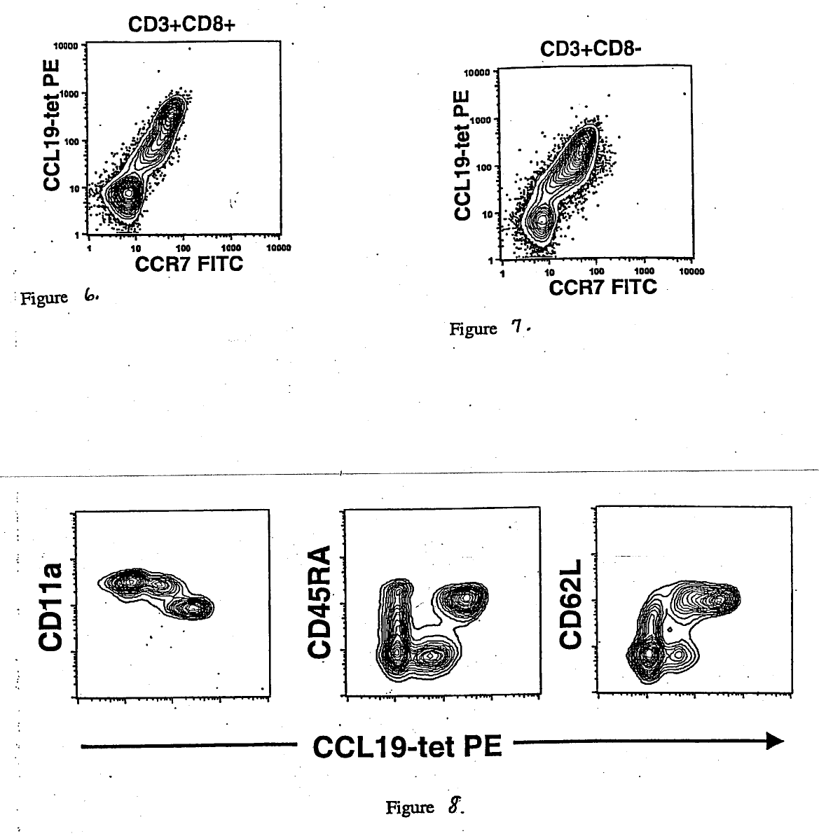

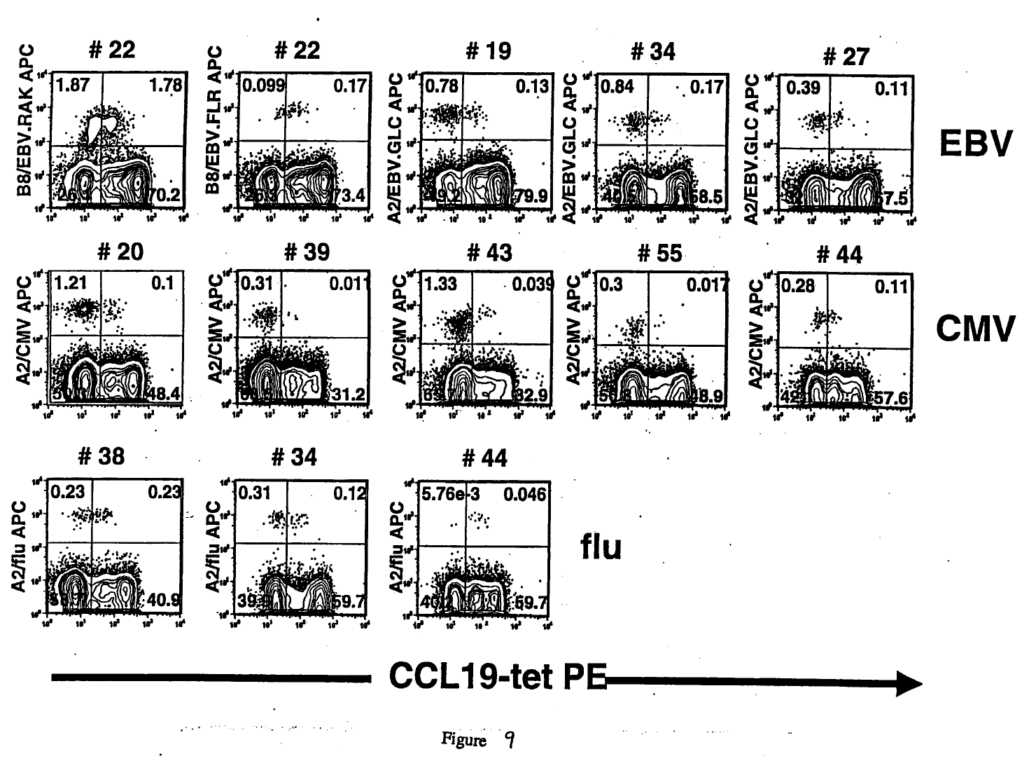

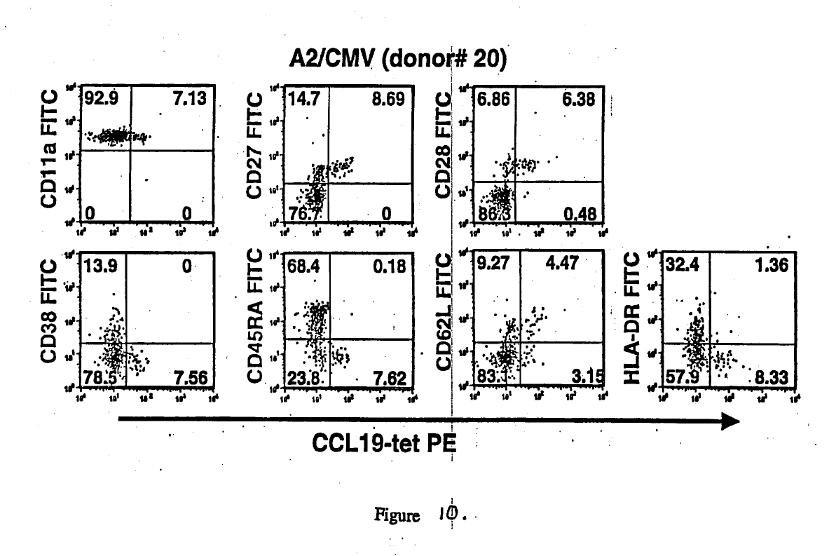

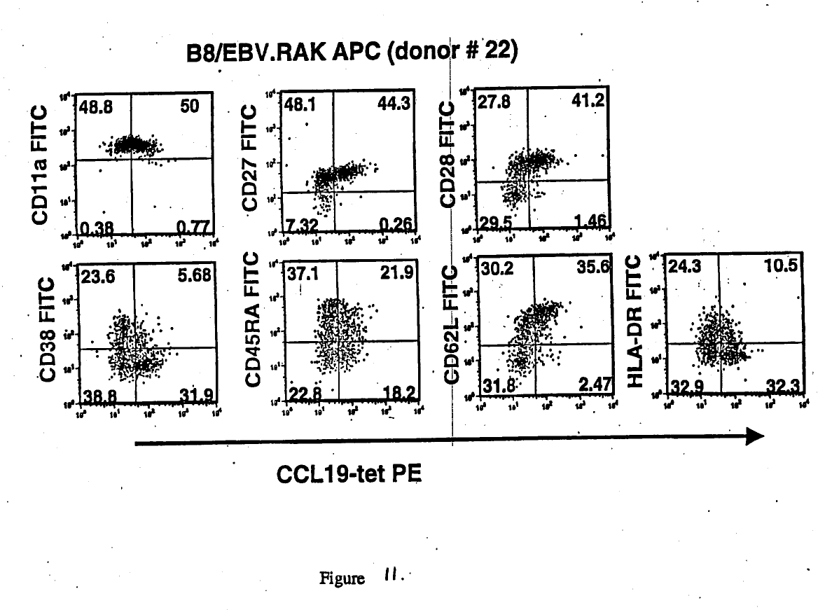

Although migration of lymphocytes toward CCL19 appeared to be mediated exclusively by CCR7, it was unknown whether CCL19 was capable of binding to other chemokine receptors without triggering functional responses. To test this, PBMCs were co-stained with a CCL19 chemotetramer and a CCR7 mAb. When both reagents were added to the cells at the same time, they both bound to the same subsets of CD3⁺CD8⁺ and CD3⁺CD8⁻ cells (FIGS. 6 and 7). In contrast, preincubation of PBMC with increased concentrations of CCR7 mAb completely abrogated CCL19-chemotetramer binding on CD3⁺CD4⁺ and CD3⁺CD8⁺ cells (FIG. 5). Taken together, these results indicated that the CCL19-chemotetramer binding on lymphocytes was restricted only to CCR7⁺ cells. Therefore, its use in phenotypic analyses was justified.

### Example 4

### Use of the CCL19-Chemotetramer to Characterize the Phenotype of the Bulk Population of CD8⁺ T cells

CCL19-chemotetramer staining on CD3⁺CD8⁺ cells identified two subsets of cells within the CCR7⁺ population (FIG. 2). These subsets were further characterized by performing immunophenotypic analyses of bulk CD3⁺CD8⁺ cells by costaining with the CCL19-chemotetramer and a panel of markers of naïve and antigen-experienced CD8⁺ T cells. Staining with CD11a, a human T cell marker for antigen-experienced cells, identified all cells within the CCR7 intermediate (CCR7^{int}) subset as bright and those within the CCR7 high (CCR7^{hi}) subset as dull (FIG. 8). The same pattern was observed with CD95 antibody. In contrast, staining with CD45RA antibody, traditionally used as a marker of naïve T cells, showed that the CCR7^{hi} subset is exclusively CD45RA bright and the CCR7^{int} subset is predominantly CD45RA⁺, with a fraction of cells seen as CD45RA dull. Discrimination with respect to CD62L, another marker for naïve T cells, showed a somewhat similar pattern. Taken together, these results indicated that the level of CCR7 present on CD8⁺ T cells allowed discrimination between the memory population, which is CCR7^{int}, and the naïve T cell population, which is CCR7^{hi}.

There was a substantial and variable degree of heterogeneity of CCR7 expression between cells specific for different viruses and antigens, and between different donors, an observation that may be explained partly by the small number of donors sampled. The CMV-specific CD8⁺ T cells exhibited the lowest percentage of CCR7⁺ cells, while A2/flu and B8/EBV.RAK CD8⁺ T cells demonstrate the highest (˜70%, donor #44). The difference in the percentage of CCR7⁺ cells in HLA-A2 restricted epitopes appeared to be attributable to the type of viral infection (i.e., latently persistent versus lytic) rather than to the type of MHC class I restriction or an anticipated variability between donors. For example, donor #44 was positive for A2/CMV and A2/flu, and the percentage of CCR7⁺ cells among these Ag-specific cells was 13.4 and 70, respectively. Overall, in A2/CMV positive individuals, the percentage of Ag-specific cells expressing CCR7 was in the range of 2-13.4%, and in A2/flu positive cells this number was between 45-70%. In EBV-specific cells, the extent of variability in the CCR7 expression was between ˜14-60%. A2/EBV-specific CD8⁺ T cells expressed CCR7 in the range of 14-18%. In B8/EBV.FLR-specific CD8⁺ T cells, which also recognize a latent antigen, CCR7 was expressed between 45-60%. A comparable number of CCR7⁺ cells was seen in B8/EBV.RAK antigen-specific CD8⁺ T cells associated with lytic infection.

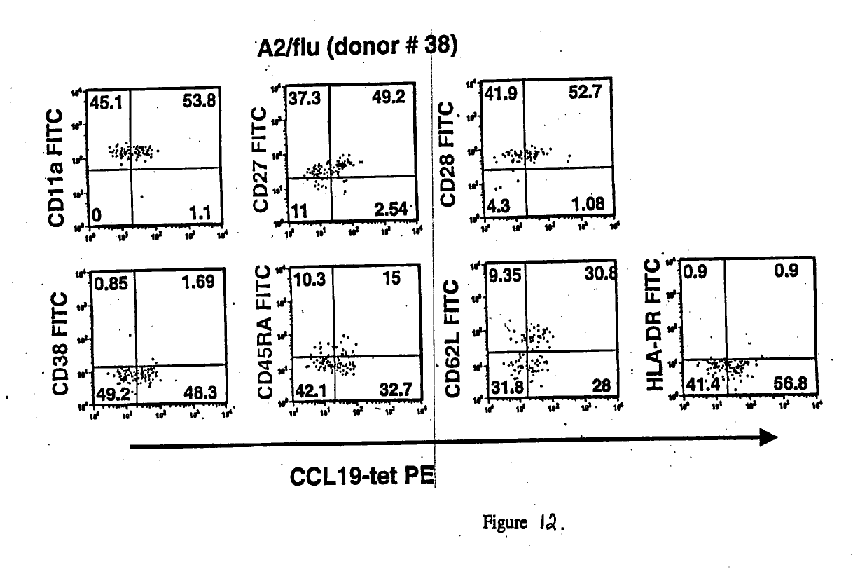

A dual pheriotypic analysis was performed for the antigen-specific CD8⁺ T cells with the CCL19-chemotetramer and a panel of cell surface markers indicating activation or memory status. FIGS. 9-12 show the results of a phenotypic analysis performed on A2/CMV, B8/EBV.RAK and A2/flu-specific CD8 T cells from different donors. The complete analysis is summarized in FIG. 25. In all but one of the donors, all the antigen-specific CD8⁺ T cells appeared to be CD11a bright. The only exception was donor #44, whose A2/flu specific cells appeared to be dull for CD11a. This was the only example to date by the hands of the authors, in which CD8⁺ T cells were defined by MHC tetramer staining that were CD11a dull in humans, rhesus macaques, or mice. Notably, A2/CMV-specific cells of donor #44 were CD 11a bright. Analysis of CD62L and CCR7 expression on the antigen-specific CD8⁺ T cells failed to reveal an absolute co-expression of these markers. For example, A2/flu antigen-specific CD8 T cells in donor #11 demonstrated 28% of CD62L⁻CCR7⁺, 9.35% of CD62L⁺CCR7⁻ and only 30% of CD62L⁺ CCR7⁺ cells. The discordant expression of CD62L and CCR7 was seen on other antigen-specific CD8 T cells as well (FIG. 25). This indicated that the lymph node homing potential of the antigen-specific CD8⁺ T cells may have been lower than that predicted from frequencies of CD62L and CCR7 alone. CCR7 was expressed on very low frequency of CD27⁻ and CL28⁻ antigen-specific CD8⁺ T cells. CD38⁺ and HLA-DR⁺ cells also did not express CCR7 at a high level. This did not hold true, however, for A2/EBV-specific cells positive for HLA-DR.

CD45RA is expressed at high frequencies on A2/CMV-specific cells (Champagne et al., Nature 410:106-111, 2001). These cells also exhibited the lowest CCR7 expression compared to all EBV and flu-specific cells. The only exception was A2/CMV cells from donor #44, whose CD45RA frequency was 22.5%. Notably, in donors #44 and AC, the expression of CCR7 on A2/CMV-specific cells was highest (13.4 and 9.4%) compared to other A2/CMV positive donors. However, there was no evidence to suggest that other antigen-specific CD8⁺ T cells exhibit an inverse correlation between CCR7 and CD45RA.

### Example 5

### CCR7⁺ Ag-Specific CD8⁺ T Cells were Capable of Secreting Effector Cytokines with Short Time Stimulation

The hypothesis of “central” and “effector” memory T cells derives from studies carried out on polyclonal populations of T cells with different antigen specificities, whose functional responses were measured using either the bacterial superantigen TSST, CD3 mAb, or PMA/ionomycin activators (Sallusto et al., Nature 401:708-712, 1999). To test this hypothesis, antigen-specific CD8⁺ T cells, stimulated in an antigen-specific manner, were examined.

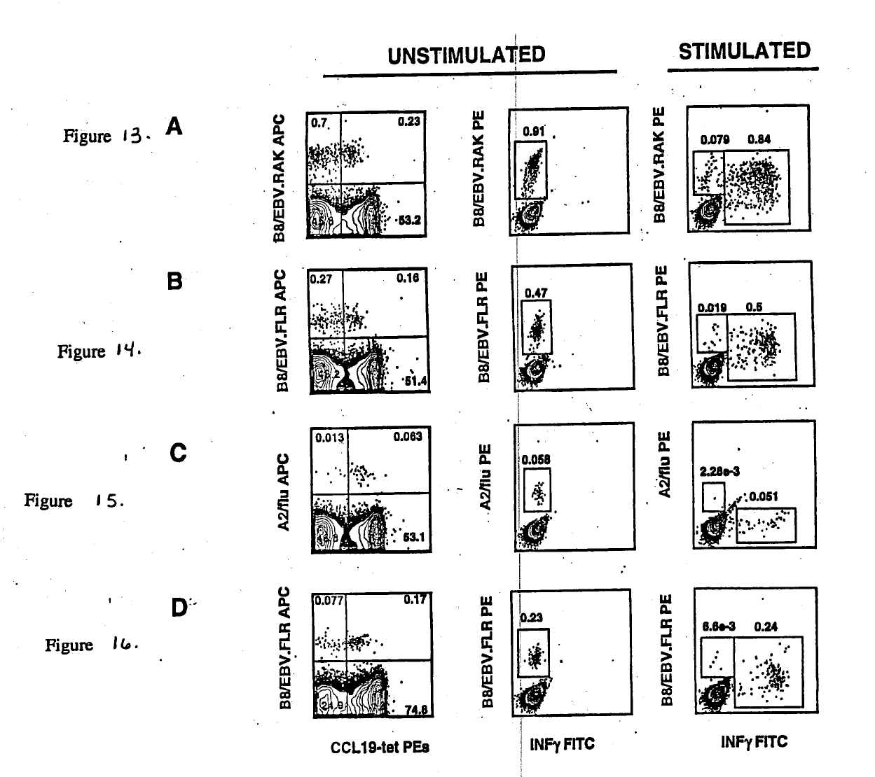

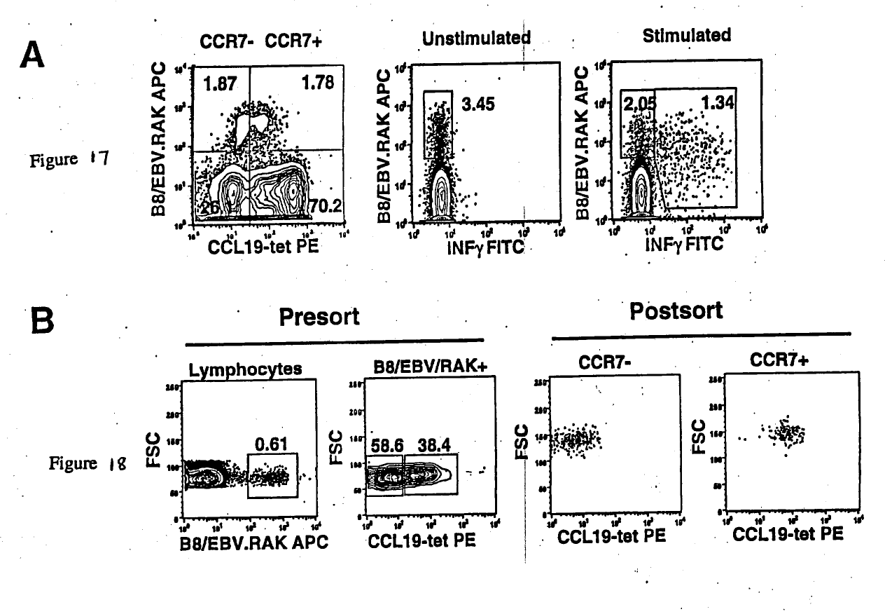

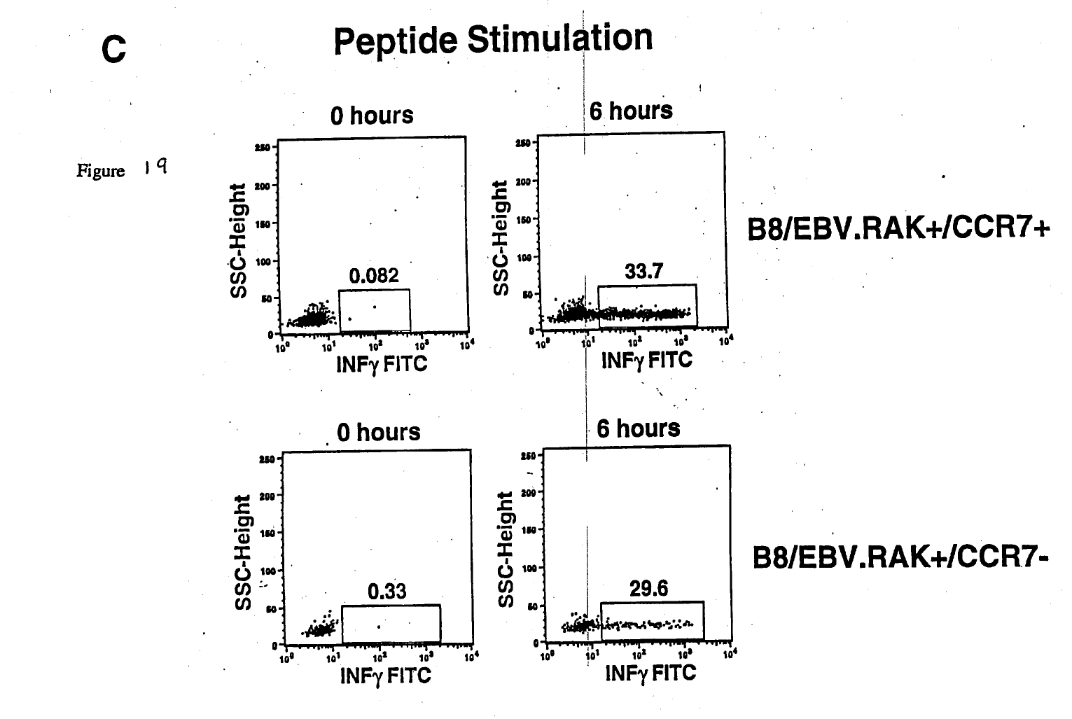

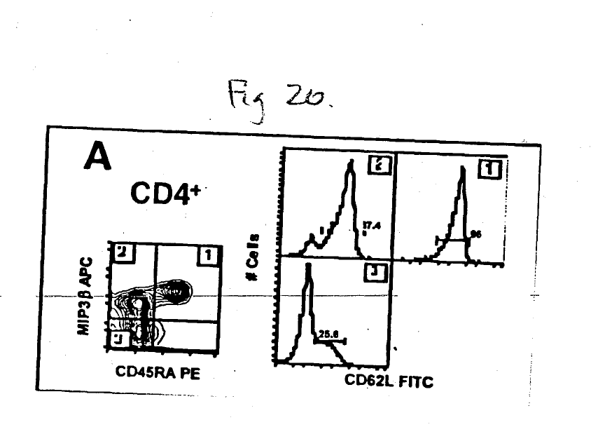

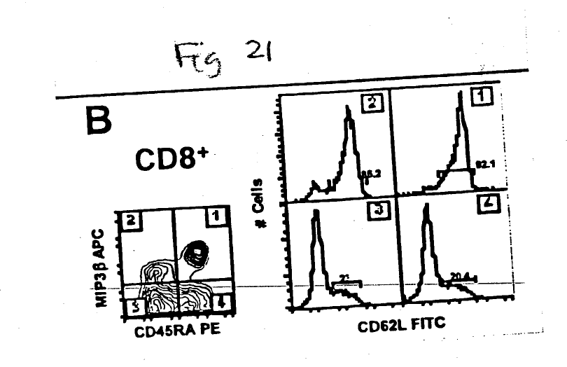

Intracellular cytokine staining for IFNγ was performed on B8/EBV.RAK, B8/EBV.FLR and A2/flu-specific CD8⁺ T cells from donor #42, following stimulation with corresponding peptides (FIG. 24) for six hours (FIGS. 13, 14 and 15). In this donor, the frequency of B8/EBV.RAK-specific CD8⁺ T cells positive for CCR7 is 0.23%. Despite this significant number of CCR7⁺ cells, nearly all of the B8/EBV.RAK-specific CD8⁺ T cells (0.84%) produced IFNγ. Similarly, IFNγ production in B8/EBV.FLR and A2/flu-specific CD8⁺ T cells of this donor after peptide stimulation was also at much higher levels than that predicted from the frequencies of CCR7⁻ cells prior to stimulation (FIGS. 14 and 15). The same pattern was observed for TNFα. This indicated that CCR7⁺ antigen-specific CD8⁺ T cells were able to express effector cytokines in a short-term antigen stimulation assay. This feature was not limited to this particular donor. IFNγ was produced in B8/EBV.FLR CD8⁺ T cells from donor #22 (FIG. 16). Here too, the frequencies of IFNγ-producing cells were much higher compared to the percentage of CCR7-negative antigen-specific CD8⁺ T cells. These results indicated that both CCR7⁺ and negative antigen-specific CD8⁺ T cells were capable of rapid expression of effector cytokines.

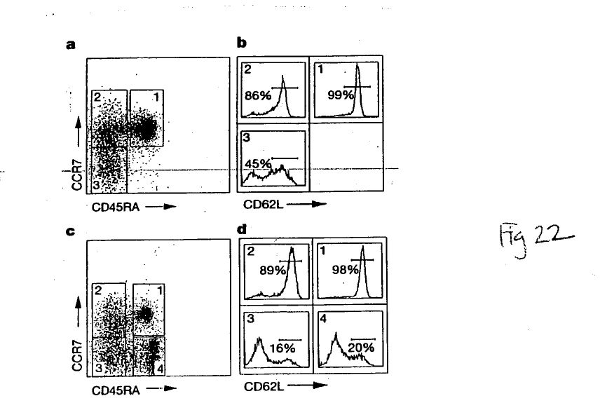

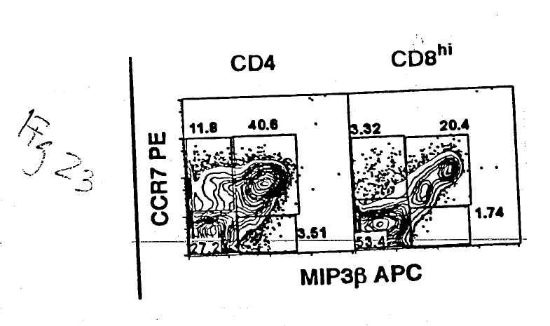

Donor #22 had a high frequency of B8/EBV.RAK-specific CD8⁺ T cells, comprising one percent of the total population of lymphocytes, and approximately 50% of the epitope-specific cells express CCR7. FIG. 17 shows the CCR7 phenotype of unstimulated B8/EBV.RAK-specific CD8 T cells from donor #22 (left panel) and the frequency of IFNγ producing cells (far right panel) after stimulation with a cognate peptide for six hours. Peptide stimulation revealed that only one third of the total B8/EBV.RAK-cell population were potent immediate producers of IFNγ (this number increased somewhat at higher peptide concentrations). To examine the relationship between the CCR7 phenotype of the unstimulated CD8⁺ T cells and the ability of these cells to produce effector cytokines, freshly prepared PBMCs from donor #22 were stained with the B8/EBV.RAK tetramer and the CCL19-chemotetramer and then FACS sorted into B8/EBV.RAK⁺CCR7⁺ and B8/EBV.RAK⁺CCR7⁻ subsets. FIG. 18 shows the quality of the sorting procedure and the gating strategy used. Subsequently, the ability of the sorted cells to produce IFNγ in a six hour stimulation assay, using peptide-pulsed autologous B-LCL as stimulators, was tested (FIG. 19). The left panel in each row shows the lack of IFNγ production prior to stimulation; panels to the right represent peptide-stimulated cells. The results indicated that both CCR7⁻ and CCR7⁺ sorted B8/EBV.RAK-specific CD8⁺ T cells expressed IFNγ at the same frequency (˜30%). The data in FIGS. 17, 18, and 19 are representative of three independent experiments. Similar results were also obtained with TNFα production. The lack of bias in the production of effector cytokines between CCR7⁻ and CCR7⁺CD8⁺ T cells indicated that cells with a surface phenotype previously ascribed to TCM were in fact capable of at least one immediate effector function, and that the heterogeneous potential of B8/EBV.RAK cells in donor #21 (FIG. 17) were not accounted for by the CCR7 phenotype.

In other studies, PBMCs were stained with an allophycocyanin (APC)-labeled huMIP-3β tetramer and a cocktail of labeled monoclonal antibodies specific for T cell-specific antigens CD45RA, CD62L, and CD4 or CD8. Analysis by flow cytometry revealed that tetramers of huMIP-3β stain the same population of cells as a CCR7 mAb (2H4) (see FIGS. 20, 21, and 22).

Human PBMC were also stained with the MIP-3β tetramer and antibodies against CD4, CD8 and CCR7. Analysis by flow cytometry revealed that at certain antibody dilution ratios, the majority of cells stained with CCR7 mAb (2H4) were also positive for MIP-3β staining (FIG. 23). At higher concentrations, the CCR7 mAb and the MIP-3β tetramer competed for binding.

### Example 6

### SUMMARY AND CONCLUSIONS

The experiments described herein were designed to test the hypothesis that the CCR7 chemokine receptor divides memory T cells into two subpopulations: a T_{CM} population (“central memory” T cells), which express CCR7, and a T_{EM} population (“effector memory” T cells), which does not. The experiments were performed by exposing antigen (Ag)-specific CD8⁺ T cells to a CCL19-chemotetramer (a fluorescent ligand-bearing complex that binds the CCR7 receptor). The CCL19 chemotetramer recognized and revealed two subsets of CCR7⁺ cells: a CCR7^{int} population containing memory cells and a CCR7^{hi} population containing naïve T cells. Phenotypic analysis of MHC class I/peptide tetramer-positive cells revealed that HLA-A2 restricted CMV-specific CD8⁺ T cells exhibit the lowest percentage of CCR7⁺ cells (0.5%), while HLA-A02 flu and HLA-B8 restricted EBV-specific CD8⁺ T cells showed the highest (45-70%). Both CCR7⁺ and CCR7⁻ antigen-specific CD8⁺ T cells produced IFNγ and TNFα following short-term peptide stimulation, and CCL19 chemotetramer specificity was verified by staining with a CCR7 mAb.

These results indicated that CCR7⁺CD8⁺ T cells exert immediate effector functions, and therefore demonstrated that the functional heterogeneity of antigen-specific CD8⁺ T cells was not correlated with the CCR7 (or CD62L) phenotype.

A number of embodiments of the invention have been described. Nevertheless, it will be understood that various modifications may be made without departing from the spirit and scope of the invention. Accordingly, other embodiments are within the scope of the following claims.

What is claimed is:

1. A multimeric complex comprising at least one receptor-binding molecule, a multivalent binding partner, and a linker, wherein the linker joins the receptor-binding molecule to the multivalent binding partner.

2. The multimeric complex of claim 1, wherein the at least one receptor-binding molecule is a growth factor, or biologically active fragment or mutant thereof.

3. The multimeric complex of claim 2, wherein the growth factor is a cytokine.

4. The multimeric complex of claim 3, wherein the cytokine is an interleukin or chemokine.

5. The multimeric complex of claim 4, wherein the interleukin is IL-1, IL-2, IL-3, IL-4, IL-5, IL-6, IL-7, IL-8, IL-9, IL-10, IL-11, IL-12, IL-13, IL-15, IL-21, interferon-alpha (IFNα), interferon-beta (IFNβ), or interferon-gamma (IFNγ).

6. The multimeric complex of claim 4, wherein the chemokine is a member of the α subfamily and/or binds a CXCR1, CXCR2, CXCR3, CXCR4, or CXCR5 receptor or is a member of the β subfamily and/or binds a CCR1, CCR2, CCR3, CCR4, CCR5, CCR6, CCR7, CCR8, CCR9, CCR10, or CCR11 molecule.

7. The multimeric complex of claim 4, wherein the chemokine is lymphotactin and/or binds a XCR1 receptor or is fractalkine and/or binds a CX₃CR1 receptor.

8. The multimeric complex of claim 4, wherein the chemokine is CCL7, CCL23, CCL27, CCL28, CXCL12, CXCL14, or CXCL15.

9. The multimeric complex of claim 2, wherein the growth factors are members of the tumor necrosis factor (TNF) family, members nerve growth factor (NGF) family, members of the transforming growth factor (TGF) family, members of the fibroblast growth factor (FGF) family, members of the insulin-like growth factor (IGF) family, members of the epidermal growth factor (EGF) family, or members of the platelet-derived growth factor (PDGF) family.

10. The multimeric complex of claim 2, wherein the growth factor is TNF, EGF, TGFα, TGFβ, FGF, NGF, erythropoietin, IGF-1, or IGF-2.

11. The multimeric complex of claim 1, wherein the at least one receptor-binding molecule is a hormone, neurotransmitter, co-stimulatory molecule or biologically active fragment or mutant thereof.

12. The multimeric complex of claim 11, wherein the hormone is a hormone produced by the adrenal gland, parathyroid gland, pituitary gland, or thyroid gland.

13. The multimeric complex of claim 11, wherein the hormone is a hormone produced by the hypothalamus, the ovary, the testicle, the pancreas, the pineal body, or the thymus.

14. The multimeric complex of claim 11, wherein the hormone is a thyroid-stimulating hormone, a follicle-stimulating hormone, a leuteinizing hormone, prolactin, growth hormone, adrenocorticotropic hormone, antidiuretic hormone, oxytocin, thyrotropin-releasing hormone, gonadotropin-releasing hormone, growth hormone-releasing hormone, corticotropin-releasing hormone, somatostatin, dopamine, melatonin, thyroxine, calcitonin, parathyroid hormone, a glucocorticoid, a mineralocorticoid, an androgen, adrenaline, an estrogen, progesterone, human chorionic gonadotropin, insulin, glucagons, somatostatin, erythropoietin, calcitriol, atrial-natriuretic peptide, gastrin, secretin, cholecystokinin, somatostatin, neuropeptide Y, ghrelin, PYY₃₋₃₆, insulin-like growth factor-1, angiotensinogen, thrombopoictin, or leptin.

15. The multimeric complex of claim 11, wherein the neurotransmitter is acetylcholine, dopamine, norepinephrine, serotonin, histamine, or epinephrine.

16. The multimeric complex of claim 15, wherein the neurotransmitter is a neuroactive peptide.

17. The multimeric complex of claim 16, wherein the neuroactive peptide is bradykinin, cholecystokinin, gastrin, secretin, oxytocin, a sleep peptide, gonadotropin-releasing hormone, beta-endorphin, enkephalin, substance P, somatostatin, prolactin, galanin, growth hormone-releasing hormone, bombesin, dynorphin, neurotensin, motilin, thyrotropin, neuropeptide Y, leuteinizing hormone, calcitonin, or vasoactive intestinal peptide.

18. The multimeric complex of claim 11, wherein the co-stimulatory molecule is B7-1 or B7-2.

19. The multimeric complex of claim 1, wherein the multivalent binding partner comprises avidin or streptavidin and the linker is a biotinylated protein.

20. The multimeric complex of claim 1, wherein the multivalent binding partner comprises a leptin and the linker is a glycosylated polypeptide.

21. The multimeric complex of claim 1, further comprising a detectable label.

22. The multimeric complex of claim 21, wherein the detectable label is joined to the multivalent binding partner, linker, or at least one receptor-binding molecule.

23. The multimeric complex of claim 21, wherein the detectable label is a fluorophor, a dye, an enzyme, or a radioisotope.

24. The multimeric complex of claim 23, wherein the fluorophor is fluorescein isothiocyanate (FITC), rhodamine, Texas Red, a green fluorescent protein, phycoerythrin (PE), or allophycocyanin (APC).

25. The multimeric complex of claim 1, wherein the complex comprises at least four receptor-binding molecules.

26. The multimeric complex of claim 25, wherein the receptor-binding molecules are chemokines.

27. The multimeric complex of claim 1, wherein the receptor-binding molecules are human receptor-binding molecules.

28. A method for detecting a ligand-binding agent, the method comprising:
(a) providing a multimeric complex comprising at least one receptor-binding molecule, a multivalent binding partner, and a linker, wherein the linker joins the receptor-binding molecule to the multivalent binding partner;
(b) exposing a receptor or an analog thereof, to the multimeric complex under conditions and for a time sufficient to allow the receptor-binding molecule to bind the receptor or the analog thereof; and
(c) determining whether the receptor-binding molecule has bound the receptor or the analog thereof, wherein binding indicates that the receptor or receptor analog is a ligand-binding agent.

29. The method of claim 28, wherein the multimeric complex comprises a complex of claim 2.

30. A method for delivering a moiety to a cell, the method comprising:
(a) providing a cell that requires the moiety, wherein the cell expresses at least one type of cell-surface receptor; and
(b) exposing the cell to a multimeric binding complex comprising at least two receptor-binding molecules that bind the type of cell-surface receptor expressed by the cell, a multivalent binding partner, a linker that joins the receptor-binding molecules to the multivalent binding partner, and the moiety required by the cell.

31. The method of claim 30, wherein the multimeric binding complex comprises a complex of claim 2.

32. The method of claim 30, wherein the moiety is a nucleic acid.

33. The method of claim 32, wherein the nucleic acid is a DNA molecule or an RNA molecule

34. The method of claim 33, wherein the RNA molecule is a single-stranded RNA molecule, a double-stranded RNA molecule, an antisense RNA molecule, or a small inhibitory RNA (siRNA) molecule, or an inhibitory RNA molecule (RNAi).

35. The method of claim 30, wherein the moiety is a cytotoxin.

36. The method of claim 35, wherein the cytotoxin is an antibody, a polysaccharide, or a chemical compound.
